(19) United States
(12) Patent Application Publication
(10) Pub. No.: US 2026/0034218 A1
(43) Pub. Date: Feb. 5, 2026

(54) ALLEVIATING GRAFT VERSUS HOST DISEASE USING ENGINEERED INKT CELLS

(71) Applicant: The Regents of the University of California, Oakland, CA (US)

(72) Inventors: Lili Yang, Los Angeles, CA (US); Yan-Ruide Li, North Hills, CA (US)

(73) Assignee: The Regents of the University of California, Oakland, CA (US)

(21) Appl. No.: 19/099,442

(22) PCT Filed: Aug. 15, 2023
(86) PCT No.: PCT/US2023/072223

§ 371 Date: Jan. 28, 2025

Related U.S. Application Data
(60) Provisional application No. 63/396,408, filed on Aug. 9, 2022.

(51) Int. Cl. A61K 40/50; A61K 40/15; A61K 40/22; A61P 37/06

(52) U.S. Cl. CPC A61K 40/50; A61K 40/15; A61K 40/22; A61P 37/06

(57) ABSTRACT

We have discovered that allogeneic HSC-engineered human iNKT (^{3rd}HSC-iNKT) cells display potent anti-GvHD functions, by eliminating antigen-presenting myeloid cells in vitro and in xenograft models, without negatively impacting tumor eradication by allogeneic T cells in preclinical models of lymphoma and leukemia. The ^{3rd}HSC-iNKT cells closely resembled the CD4⁻CD8^{-/+} subsets of endogenous human iNKT cells in phenotype and functionality. Embodiments of the invention harness these discoveries in new methods and materials for alleviating graft versus host disease.

### CROSS-REFERENCE TO RELATED APPLICATIONS

This application claims priority under Section 120 from U.S. Patent Application Ser. No. 63/398,408, filed Aug. 16, 2022, the contents of which are incorporated herein by reference.

### TECHNICAL FIELD

The present invention relates to methods and materials for alleviating graft versus host disease.

### BACKGROUND OF THE INVENTION

In 2018 alone, more than 47,000 bone marrow transplantations were performed worldwide, 19,000 (41%) of which were allogeneic and nearly all for the treatment of leukemia/lymphoma (Passweg et al., 2020). Allogeneic hematopoietic stem cell transplantation (allo-HSCT) is a curative therapy for hematologic malignancies such as leukemia/lymphoma owing to the graft-versus leukemia/lymphoma (GvL) effect elicited by alloreactive donor T cells (Appelbaum, 2001; Gribben and O'Brien, 2011; Shlomchik, 2007).

However, the development of graft-versus-host disease (GvHD) mediated by alloreactive donor T cells responding to minor or major histocompatibility antigen disparities between donor and recipient remains a major cause of patient morbidity and mortality for patients receiving T-cell replete allo-HSCT (Chakraverty and Sykes, 2007; Ferrara et al., 2009; Hill et al., 2021). T cell depletion of the graft can reduce the incidence and severity of GvHD in patients but is associated with an increased risk of graft rejection, infections, and leukemia relapse (Apperley et al., 1986). Therefore, extensive research has been focused on identifying other cellular components of the graft that could modulate donor T cells and reduce the risk and severity of GvHD without diminishing normal immunological functions, including NK (Yamasaki et al., 2003), B (Shimabukuro-Vornhagen et al., 2009), and CD4⁺CD251^{hi}FoxP3⁺ T regulatory (Treg) cells (Pabst et al., 2007; Wolf et al., 2007).

For the reasons noted above, there is a need in the art for new materials and methods useful for alleviating GvHD.

### SUMMARY OF THE INVENTION

As noted above, allo-HSCT is a curative therapy for hematologic malignancies owing to the GvL effects mediated by alloreactive T cells. However, these same T cells also mediate GvHD, a severe side effect which limits the wide-spread application of allo-HSCT therapies in the clinic. Invariant natural killer T (iNKT) cells can ameliorate GvHD while preserving GvL effect, but the clinical application of these cells is restricted by their scarcity. Here, we report the generation and new use of allogeneic (third-party) HSC-engineered human iNKT (^{3rd}HSC-iNKT) cells, using a method that combines HSC gene engineering and in vitro HSC differentiation. The ^{3rd}HSC-iNKT cells of the invention closely resemble the CD4⁻CD8^{-/+} subsets of endogenous human iNKT cells in phenotype and functionality. We have further discovered that these cells display potent anti-GvHD functions and can, for example, eliminate antigen-presenting myeloid cells in vitro and in xenograft preclinical models of lymphoma and leukemia without negatively impacting tumor eradication by allogeneic T cells. The disclosure presented herein therefore indicates that ^{3rd}HSC-iNKT cells can be used in off-the-shelf cell methods for GvHD prophylaxis and therapy.

As discussed below, the invention disclosed herein has a number of embodiments. Embodiments of the invention include, for example, methods of inhibiting or treating a graft versus host disease in a subject in need thereof, for example in a patient diagnosed with hematologic malignancy such as a leukemia or a lymphoma. These methods include administering to the subject a therapeutically effective amount of allogeneic HSC-engineered human iNKT cells (e.g., at least 0.031×10⁶ cells/kg of body weight of the allogeneic HSC-engineered human iNKT cells) so that graft versus host disease is inhibited or treated in the subject. In this context, embodiments of the invention can be used to treat acute graft-versus-host-disease (aGVHD) and/or chronic graft-versus-host-disease (cGVHD). In these methods, the engineered iNKT cells typically comprise one or more exogenous nucleic acids transduced therein such as a Vα24-Ja18 iNKT cell receptor gene, a suicide gene or the like.

In illustrative methods of the invention, the subject/patient is someone undergoing treatment for a hematologic malignancy. In typical embodiments of the invention, the subject is selected to be patient who has undergone or will undergo an allogeneic hematopoietic stem cell transplantation procedure. In certain embodiments, the subject is administered the allogeneic HSC-engineered human iNKT cells at the time the subject undergoes the allogeneic hematopoietic stem cell transplantation procedure. In certain embodiments of the invention, the subject is administered allogeneic HSC-engineered human iNKT cells mixed or in combination with allogeneic hematopoietic stem cells.

Related embodiments of the invention include methods of depleting allogeneic CD14⁺ myeloid cells from a subject transfused with allogenic leukocytes. Typically, these methods comprise administering to said subject amounts of allogeneic HSC-engineered human iNKT cells sufficient to target the allogeneic CD14⁺ myeloid cells in the subject, thereby depleting the HSC-engineered human iNKT cells in the subject. In illustrative embodiments of the invention, the HSC-engineered human iNKT cells comprise one or more exogenous nucleic acids that includes a T cell receptor gene (e.g., a iNKT receptor gene such as Vα24-Jα18, or a classical α or β T cell receptor gene), and/or a suicide gene, and/or a gene encoding a polypeptide that promotes growth or a function of the HSC-engineered human iNKT cells.

Embodiments of the invention also include methods of inhibiting or suppressing expansion of Th1-type pathogenic donor T cells in a subject (e.g. a patient diagnosed with hematologic malignancy) treated with allogenic T cells in a therapeutic regimen, the methods comprising administering to the subject amounts of allogeneic HSC-engineered human iNKT cells sufficient to inhibit or suppress the expansion of Th1-type pathogenic donor T cells in the subject (e.g., at least 1×10⁶ allogeneic HSC-engineered human iNKT cells or at least 0.031×10⁶ cells/kg of body weight of the allogeneic HSC-engineered human iNKT cells). In certain embodiments, the subject is administered allogeneic HSC-engineered human iNKT cells at the time that the subject is treated with the allogenic T cells in the therapeutic regimen.

In typical embodiments of the invention, the allogeneic HSC-engineered human iNKT cells used in the methods are derived from hematopoetic stem cells transduced with an exogenous nucleic acid comprising a iNKT receptor such as a Vα24-Jα18 T cell receptor gene. In certain embodiments, HSC-engineered human iNKT cells are lacking or have reduced surface expression of at least one HLA-1 or HLA-II molecule. In some embodiments, the lack of surface expression of HLA-I and/or HLA-II molecules is achieved by disrupting the genes encoding individual HLA-I/II molecules, or by disrupting the gene encoding B2M (beta 2 microglobulin) that is a common component of all HLA-I complex molecules, or by disrupting the genes encoding CIITA (the class II major histocompatibility complex transactivator) that is a critical transcription factor controlling the expression of all HLA-II genes.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications. The sections below disclose and describe further aspects, methods and/or materials in connection with the invention disclosed herein. Certain aspects of the invention are also shown in Li et al., iScience volume 25, issue 9, 104859, Sep. 16, 2022 (hereinafter “Li et al.”) the contents of which are incorporated by reference.

### BRIEF DESCRIPTION OF THE DRAWINGS

FIG. 1: Ex vivo generation and characterization of HSC-engineered iNKT (HSC-iNKT) cells. (A) Experimental design. HSC, hematopoietic stem cell; CB, cord blood; αGC, α-galactosylceranmde; Lenti/iNKT-sr39TK, lentiviral vector encoding an iNKT TCR gene and an sr39TK suicide/PET imaging gene; ATO, artificial thymic organoid; CMC, chemistry, manufacturing, and controls; MOA, mechanism of action. (B and C) FACS monitoring of HSC-iNKT cell development during the 2-stage Ex Vivo HSC-iNKT Cell Culture. iNKT cells were identified as iNKT TCR⁺ TCRαβ⁺ cells. iNKT TCR was stained using a 6B11 monoclonal antibody. (B) Generation of HSC-iNKT cells using an ATO approach. (C) Generation of HSC-iNKT cells using a Feeder-Free approach. (D) Table summarizing the production of HSC-iNKT cells. (E) FACS detection of surface markers, intracellular cytokines, and cytotoxic molecules of HSC-iNKT cells. Healthy donor periphery blood mononuclear cell (PBMC)-derived conventional αβ T (PBMC-Tcon) and iNKT (PBMC-iNKT) cells were included for comparison. Representative of over 10 experiments.

FIG. 2: Third-party HSC-iNKT (^{3rd}HSC-iNKT) cells ameliorate graft-versus-host disease (GvHD) in NSG mice engrafted with donor-mismatched human PBMCs. (A-F) Sublethally irradiated NSG mice received intravenous injection of 2×10⁷ random healthy donor PBMCs with or without the addition of 2×10^{7 3rd}HSC-iNKT cells and were then observed for GvHD development. N=10. (A) Experimental design. (B) Clinical GvHD score (p was calculated using data on day 40). A clinical GvHD score was calculated as the sum of individual scores of 6 categories (body weight, activity, posture, skin thickening, diarrhea, and dishevelment; score 0-2 for each category). (C) Body weight (p was calculated using data on day 40). (D) Kaplan-Meier survival curves. (E) FACS detection of human T cells in peripheral blood. (F) Representative image of experimental mice on day 40. (G-J) Histological analyses of GvHD target organs (i.e., lung, liver, salivary glands, and skin) of experimental mice analyzed 40 days following PBMC inoculation. N=5-6. (G) H&E-stained tissue sections. Scale bar: 100 μm. (H) Quantification of (G). (I) Human CD3 antibody-stained tissue sections. Scale bar: 100 μm. (J) Quantification of (1). Representative of 3 experiments. All data are presented as the mean±SEM. ns, not significant, *p<0.05, **p<0.01, ***p<0.001, and ****p<0.0001 by Student's t test (B, C, E, H, and J) or by log rank (Mantel-Cox) test adjusted for multiple comparisons (D).

FIG. 3: ^{3rd}HSC-iNKT cells ameliorate GvHD through rapid depletion of donor CD14⁺ myeloid cells that exacerbate GvHD. (A-C) Sublethally irradiated NSG mice received intravenous injection of 2×10⁷ healthy donor PBMCs with or without the addition of 2×10^{7 3rd}HSC-iNKT cells and were sacrificed 3 days later. (A) Experimental design. (B) FACS detection of CD14⁺ myeloid cells in the lymphohematopoietic system (i.e., blood, spleen and lymph nodes) and GvHD target organs (i.e., liver and lung). (C) Quantification of (B). N=4. (D-H) Sublethally irradiated NSG mice received intravenous injection of 9×10⁶ CD14-depleted healthy donor PBMCs (matching T cell number to 2×10⁷ non-CD14-depleted PBMCs) with or without the addition of 2×10^{7 3rd}HSC-iNKT cells and were then observed for GvHD development. (D) Experimental design. (E) Clinical GvHD score. (F) Body weight. (G) Kaplan-Meier survival curves. (H) Human T cells in peripheral blood. N =8. Representative of 2 experiments. All data are presented as the mean±SEM. ns, not significant, *p<0.05, **p<0.01, ***p<0.001, and ****p<0.0001 by Student's ttest (C, E, F and H) or by log rank (Mantel-Cox) test adjusted for multiple comparisons (G).

FIG. 4: ^{3rd}HSC-iNKT cells ameliorate GvHD through eliminating donor CD14⁺ myeloid cells through CD1d recognition. In vitro mixed lymphocyte reaction (MLR) assay was performed using healthy donor PBMCs (responders) co-cultured with irradiated donor-mismatched allogeneic PBMCs (stimulators) with or without the addition of ^{3rd}HSC-iNKT cells. Where applicable, purified anti-human CD1d antibody or its IgG isotype control was also added. In order to identify responders and stimulators by flow cytometry, HLA-A2⁺ responders and HLA-A2⁻ stimulators were used in the study. (A) Experimental design. (B) ELISA analyses of IFN-γ production in the indicated MLR co-cultures. Supematant were collected and analyzed on day 4. N=3. (C) FACS analyses of CD1d expression on the indicated cells. N=4. (D) FACS detection of T, B, and CD14⁺ cells of responders in multiple MLR assays one day after MLR co-culture. (E) Quantification of (D). N=4. Representative of 3 experiments. All data are presented as the mean SEM. ns, not significant, **p<0.01, ***p<0.001, and ****p<0.0001 by one-way ANOVA.

FIG. 5: ^{3rd}HSC-iNKT cells ameliorate GvHD while preserving GvL in a human B cell lymphoma xenograft NSG mouse model. Sublethally irradiated NSG mice were inoculated with 1×10⁵ Raji-FG cells, followed by intravenous injection of 2×10⁷ healthy donor PBMCs with or without the addition of 2×10^{7 3rd}HSC-iNKT cells. Mice were monitored for tumor burden and GvHD development. Raji-FG, human B cell lymphoma Raji cell line engineered to overexpress firefly luciferase and green fluorescence protein (FG) dual reporters. BLI, bioluminescence imaging. (A) Experimental design. (B) BLI images showing tumor loads in experimental mice over time. (C) Quantification of (B). (D) Clinical GvHD score (p was calculated using data on day 40). (E) Body weight (p was calculated using data on day 36). (F) Kaplan-Meier survival curves. (G) Human T cells in peripheral blood of experimental mice over time. N=10. Representative of 2 experiments. All data are presented as the mean±SEM. ****p<0.0001 by Student's t test (D, E, G), one-way ANOVA (C), or by log rank (Mantel-Cox) test adjusted for multiple comparisons (F).

FIG. 6: ^{3rd}HSC-iNKT cells ameliorate GvHD while preserving GvL in a human acute myeloid leukemia (AML) xenograft NSG mouse model. Sublethally irradiated NSG mice were inoculated with 2×10⁵ HL60-FG human AML cells, followed by intravenous injection of 2×10⁷ healthy donor PBMCs with or without the addition of 2×10^{7 3rd}HSC-iNKT cells. Mice were monitored for tumor burden and GvHD development. HL60-FG, human AML HL60 cell line engineered to overexpress FG dual reporters. (A) Experimental design. (B) BLI images showing tumor loads in experimental mice over time. (C) Quantification of (B). (D) Clinical GvHD score (p was calculated using data on day 40). (E) Body weight. (F) Kaplan-Meier survival curves. (G) Human T cells in peripheral blood of experimental mice over time. N=10. Representative of 2 experiments. All data are presented as the mean ±SEM. ***p<0.001, ****p<0.0001 by Student's t test (D, G), one-way ANOVA (C), or by log rank (Mantel-Cox) test adjusted for multiple comparisons (F).

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Invariant nature killer T (iNKT) cells have been studied extensively for their roles in modulating GvHD and GvL. iNKT cells are a small subset of αβ T cells that express both a semi-invariant T cell receptor (Vα24-Jα18 in humans and Va14-Jα18 in mice paired with a limited selection of VP chains) and natural killer cell markers (e.g., CD161 in humans and NK1.1 in mice) (Bendelac et al., 2007; Brennan et al., 2013; Brigl and Brenner, 2004; Kronenberg, 2005; Kumar et al., 2017; Lantz and Bendelac, 1994; Taniguchi et al., 2003). Unlike conventional αβ TCRs that recognize peptide antigens presented on classical polymorphic MHC Class I and II molecules, the iNKT TCR recognizes glycolipid antigens presented on non-polymorphic MHC Class I-like molecule CD1d (Cohen et al., 2009). iNKT cells in mouse comprise CD4+ and CD4⁻CD8⁻ (double negative, DN) subsets (Brigi and Brenner, 2004), and iNKT cells in human comprise CD4⁺, CD8⁺ and DN subsets (Brigl and Brenner, 2004). iNKT cells express high levels of cytokine mRNA and produce large amounts of cytokines upon primary stimulation (Brigl and Brenner, 2004). iNKT cell subset have differential cytokine patterns and cytolytic functions: CD4⁺ iNKT cell subset produce much higher levels of IL-4 as compared to CD8⁺ and DN subsets; the latter subsets express much higher levels of Granzyme B and Perforin and have stronger cytolytic function as compared to the former (Brigl and Brenner, 2004).

Here, we report a new use for allogeneic (third-party) HSC-engineered human iNKT (^{3rd}HSC-iNKT) cells. The ^{3rd}HSC-iNKT cells of the invention closely resemble the CD4⁻CD8^{-/+} subsets of endogenous human iNKT cells. We have further discovered that these cells display potent anti-GvHD functions and can, for example, eliminate antigen-presenting myeloid cells in vitro and in xenograft preclinical models of lymphoma and leukemia without negatively impacting tumor eradication by allogeneic T cells.

As discussed below, the invention disclosed herein has a number of embodiments. Embodiments of the invention include, for example, methods of inhibiting or treating a graft versus host disease in a subject/patient in need thereof. These methods include administering to the subject a therapeutically effective amount of allogeneic HSC-engineered human iNKT cells (e.g., at least 0.031×10⁶ cells/kg of body weight of the allogeneic HSC-engineered human iNKT cells) such that graft versus host disease is inhibited or treated in the subject. In this context, embodiments of the invention can be used to treat acute graft-versus-host-disease (aGVHD) and/or chronic graft-versus-host-disease (cGVHD). Typically in these methods, the engineered iNKT cells comprise one or more exogenous nucleic acids that has been transduced therein. In illustrative embodiments, the one or more exogenous nucleic acids comprise a Vα24-Jα18 iNKT cell receptor gene; and/or the one or more exogenous nucleic acids comprise a classical α or β T cell receptor gene.

In certain embodiments of the invention, the subject/patient has been diagnosed with hematologic malignancy such as a leukemia or a lymphoma. In some embodiments of the invention, the subject is selected to be patient who has undergone or will undergo an allogeneic hematopoietic stem cell transplantation procedure. In certain embodiments, the subject is administered the allogeneic HSC-engineered human iNKT cells at the time the subject undergoes the allogeneic hematopoietic stem cell transplantation procedure. In some embodiments of the invention, the subject is administered allogeneic HSC-engineered human iNKT cells mixed or in combination with allogeneic hematopoietic stem cells. In some embodiments, the subject is administered at least 1×10⁶ allogeneic HSC-engineered human iNKT cells. In certain embodiments, the subject is administered at least 0.031×10⁶ cells/kg of body weight of the allogeneic HSC-engineered human iNKT cells.

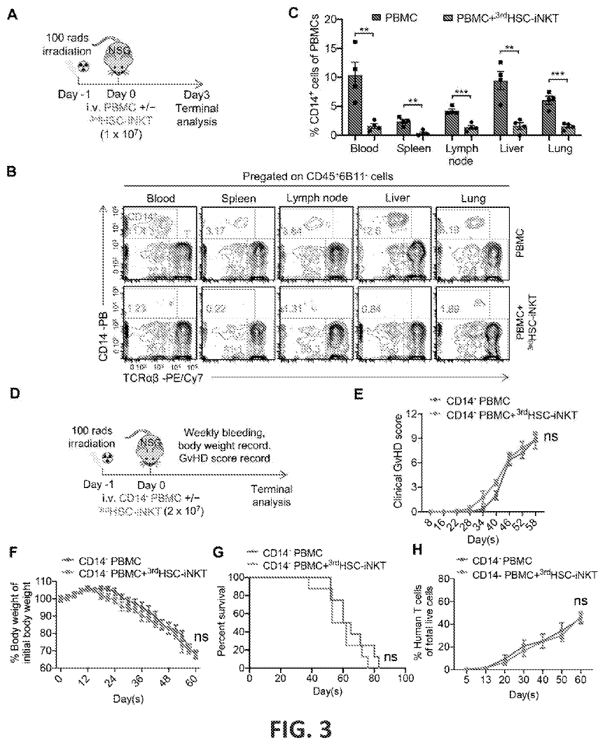

Embodiments of the invention also include methods of depleting allogeneic CD14⁺ myeloid cells from a subject transfused with allogenic leukocytes (including the allogeneic CD14⁺ myeloid cells). See, for example, the data presented in FIG. 3 and FIGS. S4A-S4E in Li et al. Typically, these methods comprise administering to said subject amounts of allogeneic HSC-engineered human iNKT cells sufficient to target the allogeneic CD14⁺ myeloid cells in the subject, thereby depleting the HSC-engineered human iNKT cells in the subject. In certain embodiments of these methods, the allogeneic HSC-engineered human iNKT cells comprise one or more exogenous nucleic acids, for example, one or more exogenous nucleic acids comprising an INKT cell receptor (e.g., a Vα24-Jα18 iNKT cell receptor gene). In certain embodiments of the invention, the subject has been diagnosed with hematologic malignancy. In some embodiments of the invention, the HSC-engineered human iNKT cells comprise one or more exogenous nucleic acids that includes a classical α or β T cell receptor gene, and/or a gene encoding a polypeptide that promotes growth or a function of the HSC-engineered human iNKT cells. In some embodiments, the subject is administered at least 1×10⁶ allogeneic HSC-engineered human iNKT cells. In certain embodiments, the subject is administered at least 0.031×10⁶ cells/kg of body weight of the allogeneic HSC-engineered human iNKT cells.

Embodiments of the invention also include methods of inhibiting or suppressing expansion of Th1-type pathogenic donor T cells in a subject (e.g., one selected to be a patient diagnosed with hematologic malignancy) treated with allogenic T cells in a therapeutic regimen. See for example the discussion below and FIGS. S2C and S2D in Li et al., In this context, Th1-type pathogenic donor T cells are well known in the art and discussed, for example in Jian et al., Front. Immunol., 5 Oct. 2021, Volume 12-2021; and Boieri et al., Exp Hematol. 2017 June;50:33-45.e3. doi: 10.1016/j.exphem.2017.02.002. Epub 2017 Feb. 24. These method comprise administering to said subject amounts of allogeneic HSC-engineered human iNKT cells sufficient to inhibit or suppress the expansion of Th1-type pathogenic donor T cells in the subject (e.g., at least 1×10⁶ allogeneic HSC-engineered human iNKT cells or at least 0.031×10⁶ cells/kg of body weight of the allogeneic HSC-engineered human iNKT cells). In typical embodiments of the invention, the allogeneic HSC-engineered human iNKT cells are derived from hematopoetic stem cells transduced with an exogenous nucleic acid comprising an iNKT cell receptor gene. In certain embodiments, the subject is administered allogeneic HSC-engineered human iNKT cells at the time that the subject is treated with the allogenic T cells in the therapeutic regimen.

In certain embodiments, HSC-engineered human iNKT cells are lacking or have reduced surface expression of at least one HLA-I or HLA-II molecule. In some embodiments, a lack of surface expression of HLA-I and/or HLA-II molecules is achieved by disrupting the genes encoding individual HLA-I/II molecules, or by disrupting the gene encoding B2M (beta 2 microglobulin) that is a common component of all HLA-I complex molecules, or by disrupting the genes encoding CIITA (the class II major histocompatibility complex transactivator) that is a critical transcription factor controlling the expression of all HLA-IL genes. See, e.g., WO 2019/241400, the contents of which are incorporated herein by reference.

Related embodiments of the invention also include, for example, methods of inhibiting or treating a graft versus host disease in a patient, by administering to the patient a therapeutically effective amount of the third-party HSC-engineered human iNKT (^{3rd}HSC-iNKT) cells disclosed herein. In certain embodiments of the invention, the patient has been diagnosed with hematologic malignancy. In typical embodiments of the invention, the patient has undergone or will undergo an allogeneic hematopoietic stem cell transplantation procedure as part of a treatment regimen for the hematologic malignancy. In certain embodiments, the patient is administered the third-party HSC-engineered human iNKT (^{3rd}HSC-iNKT) cells at the time the subject undergoes the allogeneic hematopoietic stem cell transplantation procedure. Optionally the patient is administered third-party HSC-engineered human iNKT (^{3rd}HSC-iNKT) cells mixed with allogeneic hematopoietic stem cells.

In illustrative embodiments of the invention, the patient is administered at least 1×10⁶ third-party HSC-engineered human iNKT (^{3rd}HSC-iNKT) cells. In certain embodiments, the patient is administered at least 0.031×10⁶ cells/kg of body weight of the third-party HSC-engineered human iNKT (^{3rd}HSC-iNKT) cells. In some embodiments of the invention, the engineered iNKT cells comprise one or more exogenous nucleic acids transduced therein. Optionally, for example, the one or more exogenous nucleic acids comprise a T cell receptor alpha chain gene and/or a T cell receptor alpha chain gene; and the engineered iNKT cells comprise clonal populations of cells comprising the T cell receptor alpha chain gene and/or the T cell receptor beta chain gene.

In the present disclosure, a patient/subject can be someone diagnosed with a disease or disorder. The disease or disorder may be at least one of a hemoglobinopathy, a congenital hemoglobinopathy, β-Thalessemia major™, sickle cell disease (SCD), severe aplastic anemia, Fanconi's anemia, dyskeratosis congenita, Blackfan-Diamond anemia, Thalassemia, congenital amegakaryocytic thrombocytopenia, severe combined immunodeficiency, T cell immunodeficiency, T cell immunodeficiency-SCID variants, Wiskott-Aldrich syndrome, a hemophagocytic disorder, a lymphoproliferative disorder, severe congenital neutropenia, chronic granulomatous disease, a phagocytic cell disorder, IPEX syndrome, juvenile rheumatoid arthritis, systemic sclerosis, an autoimmune disorder, an immune dysregulation disorder, mucopolysaccharoidoses, MPS-I, MPS-VI, osteopetrosis, a metabolic disease, globoid cell leukodystrophy (Krabbe), metachromatic leukodystrophy, cerebral X-linked adrenoleukodystrophy, a myelofibrosis disease, a myeloproliferative disease, a plasma cell disorder, a mast cell disease, common variable immunodeficiency, chronic granulomatous disease, multiple sclerosis, systemic sclerosis, rheumatoid arthritis, systemic lupus erythematosus, Crohn's disease and polymyositis-dermatomyositis. For example, the subject can be diagnosed with a disease or disorder and be designated to undergo an allogeneic transplant. Alternatively, the subject can be diagnosed with a disease or disorder and has previously undergone an allogeneic transplant.

In the present disclosure the subject can be diagnosed with a cancer. The cancer may be a hematological cancer. The cancer can be at least one of Acute myeloid leukemia, myelodysplastic syndrome, follicular lymphoma, diffuse large B cell lymphoma, acute lymphoblastic leukemia, multiple myeloma, Hodgkin lymphoma, chronic myeloid leukemia, T cell non-Hodgkin lymphoma, lymphoblastic B cell non-Hodgkin lymphoma (non-Burkitt), Burkitt's lymphoma, anaplastic large cell lymphoma, germ cell tumor, Ewing's sarcoma, soft tissue sarcoma, neuroblastoma, Wilms' tumor, osteosarcoma, medulloblastoma, acute promyelocytic leukemia, mantle cell lymphoma, T cell lymphoma, lymphoplasmacytic lymphoma, cutaneous T cell lymphoma, plasmablastic lymphoma, chronic lymphocytic leukemia, breast cancer and renal cancer. For example, the subject can be diagnosed with a cancer and be designated to undergo an allogeneic transplant. Alternatively, the subject can be diagnosed with a cancer and has previously undergone an allogeneic transplant.

In some embodiments of the methods and uses of the present disclosure, a subject can be diagnosed with a cancer and be designated to undergo a transplant in order to treat the cancer. In some embodiments of the methods and uses of the present disclosure, a subject can be diagnosed with a cancer and has previously undergone a transplant in order to treat the cancer.

In some embodiments of the methods and uses of the present disclosure, a subject can be diagnosed with a cancer and be designated to undergo an allogeneic transplant in order to treat the cancer. In some embodiments of the methods and uses of the present disclosure, a subject can be diagnosed with a cancer and has previously undergone an allogeneic transplant in order to treat the cancer.

In some embodiments of the methods and uses of the present disclosure, a subject can be diagnosed with a disease or disorder and be designated to undergo a transplant in order to treat the disease or disorder. In some embodiments of the methods and uses of the present disclosure, a subject can be diagnosed with a disease or disorder and has previously undergone a transplant in order to treat the disease or disorder. In some embodiments of the methods and uses of the present disclosure, a subject can be diagnosed with a disease or disorder and be designated to undergo an allogeneic transplant in order to treat the disease or disorder. In some embodiments of the methods and uses of the present disclosure, a subject can be diagnosed with a disease or disorder and has previously undergone an allogeneic transplant in order to treat the disease or disorder.

In the present disclosure, a subject can have been previously administered a transplant. Accordingly, ^{3rd}HSC-iNKT cells can be administered to the subject after the latter has been administered a transplant. In some embodiments of the methods and uses of the present disclosure, a subject can have been previously administered an allogeneic transplant. Accordingly, in some embodiments an at least one therapeutically effective amount of ^{3rd}HSC-iNKT cells can be administered to the subject after the subject has been administered an allogeneic transplant.

In some embodiments of the methods and uses of the present disclosure, a subject can have been previously administered a conditioning therapy in connection with a transplant. In some embodiments of the methods and uses of the present disclosure, a subject can have been previously administered a conditioning therapy in connection with an allogeneic transplant. In some embodiments a conditioning therapy can comprise the administration of radiation therapy, chemotherapy, radiomimetic therapy or any combination thereof. In some embodiments a radiation therapy can comprise total body irradiation.

A conditioning therapy may be administered in connection with the allogenic transplant. A conditioning therapy can comprise, such as consist of, the administration of radiation therapy, chemotherapy, radiomimetic therapy or any combination thereof. The radiation therapy can comprise total body irradiation.

The sections below disclose and describe further aspects, methods and/or materials in connection with the invention disclosed herein. Certain aspects of the invention such as supplemental materials are discussed in Li et al., iScience volume 25, issue 9, 104859, Sep. 16, 2022 (hereinafter “Li et al.”) the contents of which are incorporated by reference.

### Illustrative Materials and Methods of the Invention

The beneficial roles of iNKT cells in reducing GvHD while retaining GvL in murine allo-HSCT has been well reported (Lan et al., 2003; Pillai et al., 2007; Schneidawind et al., 2014; Zeng et al., 1999). Previous studies demonstrated that total lymphoid irradiation (TLI) conditioning prior to allo-HSCT prevented GvHD and preserved GvL effect due to the selective depletion of host conventional T cells and relative expansion of NKT cells (Lan et al., 2003; Zeng et al., 1999). The host iNKT cells interact with donor myeloid cells to augment donor Treg expansion (Pillai et al., 2007). Addition of recipient, donor, or third-party (heterologous) iNKT cells into the allograft was also shown to significantly reduce the risk of GvHD in allo-HSCT without diminishing GvL by polarizing donor T cells to a Th2 phenotype (Schneidawind et al., 2015, 2014).

The protective roles of human iNKT cells against GvHD have also been highlighted by multiple clinical studies. Non-myeloablative conditioning with TLI/anti-Thymocyte Globulin (ATG) prior to allo-HSCT coincided with a higher iNKT/T cell ratio, decreased incidences of GvHD, and retained GvL effect (Kohrt et al., 2009; Lowsky et al., 2005). Patients with GvHD early after transplantation were found to have reduced numbers of total circulating iNKT cells (Haraguchi et al., 2004), whereas enhanced iNKT cell reconstitution following allo-HSCT positively correlated with a reduction in GvHD without loss of GvL effect (Rubio et al., 2012). In particular, high CD4⁻, but not CD4⁺, iNKT cell numbers in donor allograft was associated with clinically significant reduction in GvHD in patients receiving allo-HSCT (Chaidos et al., 2012). Thus, increasing the numbers of iNKT cells, particularly the CD4⁻ iNKT cells, in the allograft may provide an attractive strategy for suppressing GvHD while preserving GvL effect. Due to their recognition of non-polymorphic CD1d (Bae et al., 2019), iNKT cells can be sourced from third-party donors.

However, human periphery blood contains extremely low number and high variability of iNKT cells (˜0.001-1% in blood), making it challenging to expand sufficient numbers of iNKT cells for therapeutic applications (Krijgsman et al., 2018). To overcome this critical limitation, we have previously established a method to generate large amounts of human iNKT cells through TCR gene engineering of hematopoietic stem cells (HSCs) followed by in vivo reconstitution; using this method, we have successfully generated both mouse and human HSC-engineered iNKT (HSC-iNKT) cells (Li et al., 2021b; Y. R. Li et al., 2022; Smith et al., 2015; Zhu et al., 2019). While such an in vivo approach to providing iNKT cells maybe suitable for autologous transplantation, applying this for allogeneic transplantation faces significant hurdles (Smith et al., 2015; Zhu et al., 2019). Here, we intended to build on the HSC-iNKT engineering approach and develop an ex vivo culture method to produce large amounts of third party human iNKT cells; these cells can potentially be used as a “universal” and “off-the-shelf” reagent for improving allo-HSCT outcomes by ameliorating GvHD while preserving GvL effect.

### Results

Ex Vivo Generation and Characterization of Human HSC-Engineered iNKT (HSC-iNKT) Cells

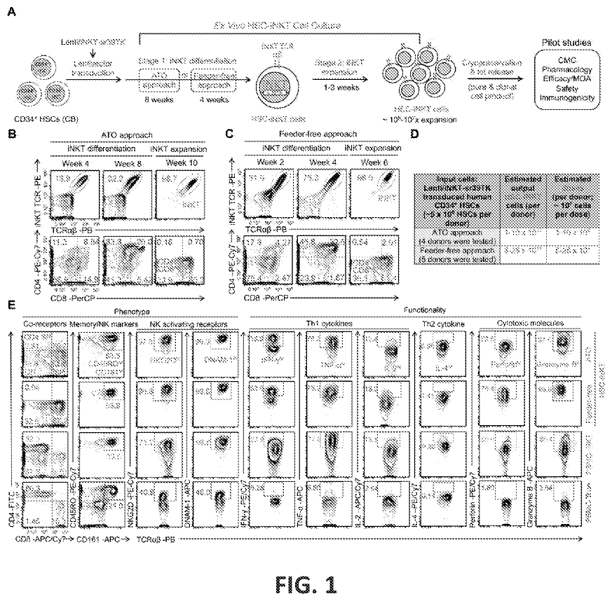

Cord blood (CB)-derived human CD34⁺ hematopoietic stem and progenitor cells (denoted as HSCs) were collected and then transduced with a Lenti/iNKT-sr39TK lentiviral vector that encodes three transgenes: a pair of iNKT TCR α and β chain genes as well as an sr39TK suicide/imaging report gene (FIG. S1A in Li et al.) (see also Li et al., 2021b; Y. R. Li et al., 2022; Zhu et al., 2019). The transduced HSCs were put into an Ex Vivo HSC-Derived iNKT (HSC-iNKT) cell culture, using either an Artificial Thymic Organoid (ATO) approach or a Feeder-Free approach (FIG. 1A). ATO culture utilizes a MS5 mouse stromal cell line overexpressed delta-like canonical Notch ligand 1 (DLL1)- or 4 (DLL4) and supports robust ex vivo differentiation and maturation of human T cells from HSCs (Li et al., 2021b; Montel-Hagen et al., 2019; Seet et al., 2017); Feeder-Free culture adopts a system of plate-bound DLL4 and vascular cell adhesion protein 1 (VCAM-1) to induce T cell commitment from HSCs (Huijskens et al., 2014; Iriguchi et al., 2021; Y. R. Li et al., 2022; Shukla et al., 2017; Themeli et al., 2013). The gene-engineered HSCs efficiently differentiated into iNKT cells in the ATO or Feeder-Free cultures system (Stage 1) over 8 weeks or 4 weeks, respectively, with over 100-fold expansion in cell numbers (FIGS. 1A-1C). These engineered HSC-iNKT cells were further expanded with irradiated PBMCs loaded with αGC, a synthetic agonist glycolipid ligand that specifically activate iNKT cells, for another 2-3 weeks (Stage 2) (FIGS. 1A-1C), resulting in another 100-1000-fold expansion of HSC-iNKT cells with >98% purity (FIGS. 1A and 1D). During the Ex Vivo HSC-iNKT cell cultures, HSC-iNKT cells followed a typical human iNKT cell development path defined by CD4/CD8 co-receptor expression (Godfrey and Berzins, 2007): HSC-iNKT cells transitioned from CD4⁻CD8⁻ to CD4⁺CD8⁺, then to CD4⁻CD8±1⁻ (FIGS. 1B and 1C). At the end of cultures, over 98% of the HSC-iNKT cells displayed a CD4⁻CD8^{+/-} phenotype (FIGS. 1B and 1C).

This manufacturing process of generating HSC-iNKT cells was robust and of high yield and high purity for all 9 donors tested (4 for ATO culture and 5 for Feeder-Free culture) (FIG. 1D). Based on the results, it was estimated that from one quality CB donor (comprising about 1-5×10⁶ HSCs), about 10¹¹-10¹² HSC-iNKT cells could be generated that can potentially be formulated into about 10,000-100,000 doses, assuming about 107 HSC-iNKT cells per dose (FIG. 1D). The dosage (about 107 HSC-iNKT cells per dose) was estimated based on an earlier clinical study, wherein 0.031×10⁶ CD4⁻ iNKT cells/kg of body weight was associated with amelioration of GvHD (Chaidos et al., 2012).

To increase the safety profile of HSC-iNKT cell product, we included an sr39TK PET imaging/suicide gene in the lentiviral vector, which allows for the in vivo monitoring of these cells using PET imaging and the elimination of these cells through ganciclovir (GCV)-induced depletion in case of an adverse event (FIGS S1A and 1B in Li et al.). In cell culture, GCV treatment induced effective killing of HSC-iNKT cells (FIGS. S1B and 1C in Li et al.). In an NSG mouse xenograft model, GCV treatment induced efficient depletion of HSC-iNKT cells from all tissues examined (i.e., blood, liver, spleen and lung) (FIGS. S1D-S1F in Li et al.). Therefore, the engineered HSC-iNKT cell product is equipped with a potent “kill switch”, significantly enhancing its safety profile.

We next studied the phenotype and functionality of HSC-iNKT cells, in comparison with healthy donor periphery blood mononuclear (PBMC)-derived iNKT (PBMC-iNKT) cells and conventional αβ T (PBMC-Tcon). HSC-iNKT cells displayed a phenotype closely resembling PBMC-iNKT cells and distinct from PBMC-Tcon cells: they expressed high levels of memory T cell markers (i.e., CD45RO) and NK cell markers (i.e., CD161, NKG2D, and DNAM-1) and expressed exceedingly high levels of Th1 cytokines (i.e., IFN-γ, TNF-α, and IL-2) as well as high levels of cytotoxic molecules (i.e., Perforin and Granzyme B) (FIG. 1E). Notably, HSC-iNKT cells produced high levels of Th1 cytokines (i.e, IFN-γ, TNF-α, and IL-2) while low levels of Th2 cytokines (i.e., IL-4), suggesting a function like that of the endogenous CD8⁺ and DN human iNKT subsets, agreeing with the CD4⁻CD8⁺⁻⁻ phenotype of these HSC-iNKT cells (FIGS. 1B, 1C, and 1E) (Li et al., 2021b; Y. R. Li et al., 2022; Zhu et al., 2019).

Third Party HSC-iNKT (^{3rd}HSC-iNKT) Cells Ameliorate Xeno-GvHD in NSG Mice Engrafted with Human PBMC

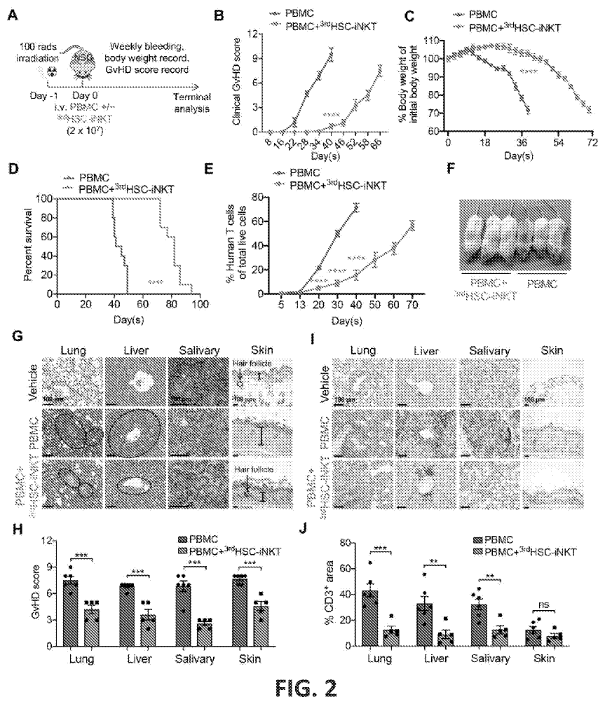

The engineered HSC-iNKT cells were predominantly CD4⁻ (FIGS. 1B, 1C, and 1E); this subset of human iNKT cells were reported to be associated with reduced GvHD in patients (Chaidos et al., 2012). To test the anti-GvHD potential of ^{3rd}HSC-iNKT cells, we utilized a xeno-GvHD model wherein NSG mice were engrafted with human PBMCs (Shultz et al., 2007). NSG mice were preconditioned with non-lethal total body irradiation (TBI, 100 cGy), and were injected intravenously (i.v.) with healthy donor PBMCs with or without the addition of ^{3rd}HSC-iNKT cells. The recipients were monitored daily for clinical signs of GvHD (FIG. 2A). The addition of ^{3rd}HSC-iNKT cells significantly delayed GvHD onset, reduced bodyweight-loss and prolonged survival (FIGS. 2B-2D and 2F). The delayed onset and reduced GvHD severity were associated with the delay of donor T cell expansion in the peripheral blood of experimental mice (FIG. 2E).

To further characterize the changes in GvHD severity, the acute and chronic GvHD overlapping target organs (i.e., lung, liver and skin) and chronic GvHD prototypical target organs (i.e., salivary glands) were collected for pathological analysis on day 40 after engrafting donor PBMCs alone or together with ^{3rd}HSC-iNKT cells (Wu et al., 2013). Compared with control NSG mice, the recipient mice engrafted with PBMCs alone showed severe infiltration and damage in the liver and lung. Although the skin tissue did not have severe infiltration, there was an enlarged epidermis, a sign of excessive collagen deposition (Wu et al., 2013). The salivary gland also showed infiltration and damage of gland follicles (FIGS. 2G-2J). The results suggested that by day 40 after PBMC engraftment, the recipient mice had overlapping acute and chronic GvHD. On the other hand, the mice receiving additional ^{3rd}HSC-iNKT cells showed marked reduction in T cell infiltration in the liver, lung and salivary gland as well as tissue damage scores (FIGS. 2G-2J). Addition of ^{3rd}HSC-iNKT cells also markedly reduced hair loss and epidermis enlargement, although T cell infiltration in the skin tissues was mild and no significant difference was observed between recipient mice with or without the addition of ^{3rd}HSC-iNKT cells (FIGS. 2G-2J).

Flow cytometry analysis also revealed significantly less numbers of donor T cells in the blood and spleen, as well as less T cell infiltration in GvHD target organs (i.e., lung, liver and bone marrow; FIGS. S2A and S2B in Li et al.). Furthermore, intracellular cytokine staining showed that by day 40 after PBMC injection, the addition of ^{3rd}HSC-iNKT cells significantly reduced the proportion of donor CD4⁺ T cells actively producing Th1-type pro-inflammatory cytokines (i.e., IFN-γ and GM-CSF); the proportion of CD4⁺ T cells producing the Th2-type anti-inflammatory cytokine (i.e., IL-4) was not changed (FIGS. S2C and S2D in Li et al.). Together, these results suggest that ^{3rd}HSC-iNKT cells suppress the expansion of Th1-type pathogenic donor T cells in target tissues and thereby ameliorating acute and chronic GvHD.

^{3rd}HSC-iNKT cells eliminate donor CD14⁺ myeloid cells in part through CD1d recognition

Donor myeloid cell-derived antigen presenting cells have been reported to exacerbate acute and chronic GvHD induced by donor T cells (Anderson et al., 2005; Chakraverty and Sykes, 2007; Jardine et al., 2020). Donor T cell production of GM-CSF has also been reported to recruit donor myeloid cells, which in turn amplifies the activation of allogeneic T cells and exacerbates GvHD severity (Piper et al., 2020; Tugues et al., 2018). Consistently, we observed that removal of CD14⁺ myeloid cells in the PBMCs reduced xeno-GvHD in NSG recipient mice (FIGS. 3A-3H). In contrast, co-injection of donor PBMCs together with ^{3rd}HSC-iNKT cells resulted in a dramatic reduction of donor CD14⁺ myeloid cells in recipient mice within three days of injections, in tissues spanning blood, lymphoid tissues (i.e, spleen and lymph node), and GvHD target tissues (i.e., liver and lung) (FIGS. 3A-3C). Meanwhile, donor T and B cell, which expressed lower levels of CD1d compared to CD14⁺ myeloid cells, showed no detectable changes (FIGS. S4A-S4E in Li et al.).

iNKT cells have been shown to target myeloid (i.e., tumor-associated 10 macrophages) and myelomonocytic cells (Cortesi et al., 2018; Gorini et al., 2017; Janakiram et al., 2017; Y.-R. Li et al., 2022; Song et al., 2009). To validate that the ^{3rd}HSC-iNKT cells ameliorate GvHD via depleting donor CD14⁺ myeloid cells in the xeno-GvHD model, we conducted another experiment wherein NSG mice received CD14⁺ myeloid cell-depleted PBMCs with or without the addition of ^{3rd}HSC-iNKT cells (FIG. 3D). Indeed, pre-depletion of CD14⁺ myeloid cells abrogated the anti-GvHD effect of ^{3rd}HSC-iNKT cells (FIGS. 3E-3H).

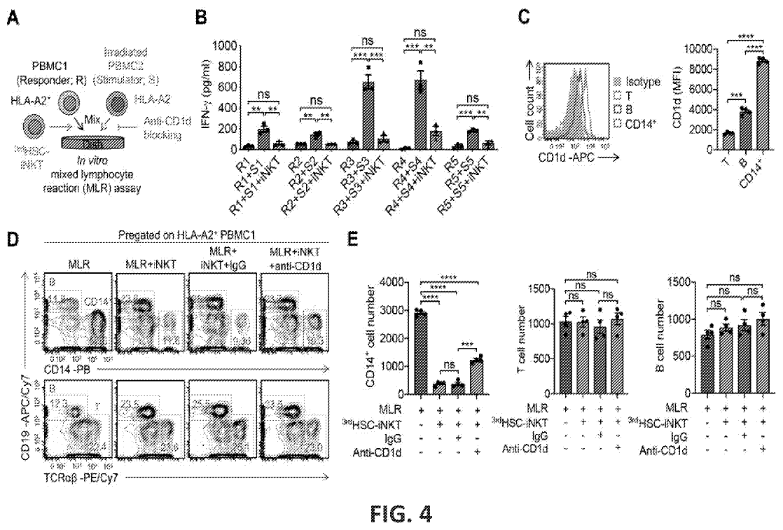

To study the molecular regulation of ^{3rd}HSC-iNKT cell deletion of donor CD14⁺ myeloid cells, we performed an in vitro mixed lymphocyte reaction (MLR) assay (FIG. 4A). Healthy donor PBMCs (non-irradiated; as responder representing donor cells) were mixed with donor-mismatched PBMCs (irradiated; as stimulator representing recipient cells) to study alloreaction (Li et al., 2021b), with or without the addition of ^{3rd}HSC-iNKT cells. A pair of HLA-A2 positive and negative PBMCs were used to distinguish responders from stimulators (FIG. 4A). In agreement with the in vivo results, in the MLR assay ^{3rd}HSC-iNKT cells effectively ameliorated alloreaction as evidenced by the reduction of IFN-γ production (FIG. 4B). Responder PBMCs contained CD14⁺ myeloid cells expressing high levels of CD1d molecule that can be recognized by iNKT TCR (Bae et al., 2019; King et al., 2018; Y. R. Li et al., 2022), corresponding with their efficient depletion by ^{3rd}HSC-iNKT cells (FIGS. 4C-4E, S4F, and S4G in Li et al.). On the other hand, human T and B cells from responder PBMCs expressed low levels of CD1d and were not altered by the addition of ^{3rd}HSC-iNKT cells (FIGS. 4C-4E). Depletion of CD14⁺ myeloid cells population was significantly alleviated by the addition of anti-CD1d blocking antibody (FIGS. 4D, 4E, S4F, and S4G in Li et al.). Taken together, ^{3rd}HSC-iNKT cells ameliorate GvHD through eliminating donor CD14⁺ myeloid cells at least partly through CD1d recognition.

^{3rd}HSC-iNKT Cells Preserved GvL Activity while Ameliorating GvHD

Next, we studied the potential of ^{3rd}HSC-iNKT cells to preserve graft-versus-leukemia (GvL) while ameliorate GvHD, using a human Raji B cell lymphoma and a human HL60 acute myeloid leukemia (AML) xenograft NSG mouse models. We engineered Raji and HL60 tumor cells to overexpress the firefly luciferase and EGFP dual-reporters (denoted as Raji-FG and HL60-FG, respectively) to enable the convenient measurement of tumor killing using in vitro luminescence reading or in vivo bioluminescence imaging (BLI). When co-cultured in vitro, ^{3rd}HSC-iNKT cells effectively killed the Raji-FG and HL60-FG cells via a NK activating receptor (i.e., NKG2D and DNAM-1)-mediated tumor targeting mechanism (FIGS. S5A-S5F in Li et al.).

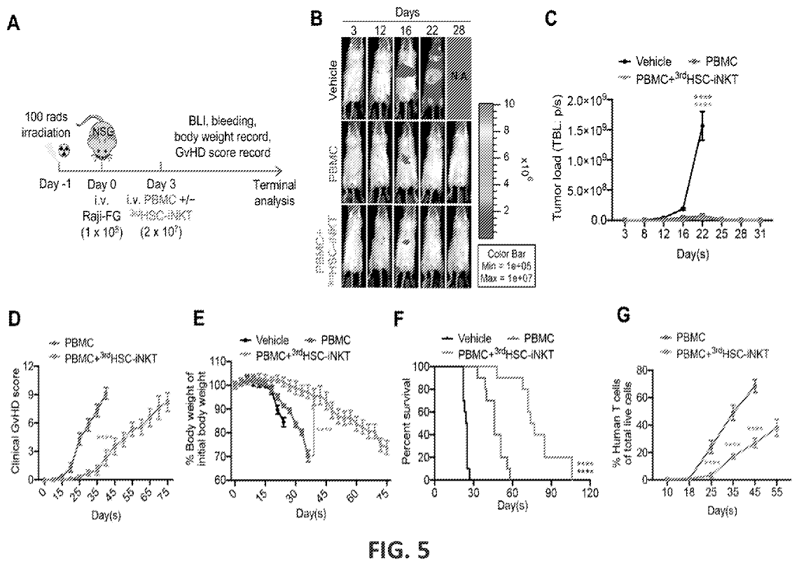

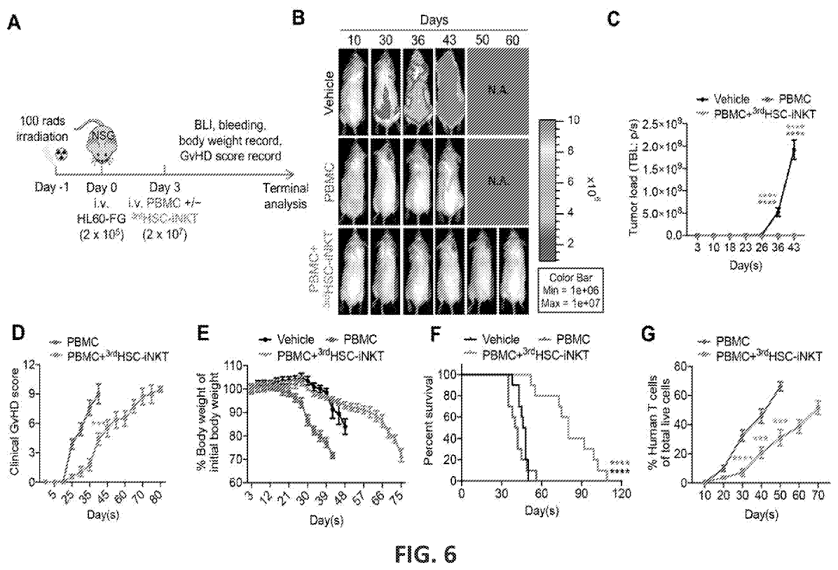

NSG mice were inoculated intravenously (i.v.) with Raji-FG cells, followed by adoptive transfer of healthy donor PBMCs without or with the addition of ^{3rd}HSC-iNKT cells (FIG. 5A). Control NSG mice receiving Raji-FG cells alone died as a result of high tumor burden by day 27 (FIGS. 5B-5F). Tumor-bearing NSG mice receiving PBMCs with or without the addition of ^{3rd}HSC-iNKT cells showed rapid clearance of the Raji-FG cells (FIGS. 5B and 5C). However, the tumor-eradicated NSG mice receiving PBMCs all died by day 58 with high clinical GvHD scores, rapid weight loss, and rapid expansion of donor T cells (FIGS. 5D-5G). The mice receiving PBMCs together with ^{3rd}HSC-iNKT cells survived significantly longer, for up to 106 days with a much slower progression of GvHD and decline in weight (FIGS. 5D-5G). Similar results were obtained from the human HL60 AML xenograft NSG mouse model (FIGS. 6A-6G). Taken together, these results strongly support the potential of ^{3rd}HSC-iNKT cells to ameliorate GvHD while preserving GvL effect in the treatment of blood cancers.

### Experimental Model and Subject Details

Mice

NOD·Cg-Prkdc^{SCID}II2rg^{tmlwjl}/SzJ (NOD/SCID/IL-2Rγ^{-/-}, NSG) mice were maintained in the animal facilities at the University of California, Los Angeles (UCLA). Six- to ten-week-old mice were used for all experiments unless otherwise indicated. All animal experiments were approved by the Institutional Animal Care and Use Committee of UCLA.

### Cell Lines and Viral Vectors

The murine bone marrow derived stromal cell line MS5-DLL4 was obtained from Dr. Gay Crooks' lab (UCLA). Human Raji B cell lymphoma cell line, HL60 acute myeloid leukemia cell line, and HEK 293T cell line were purchased from the American Type Culture Collection (ATCC).

Lentiviral vectors used in this study were all constructed from a parental lentivector pMNDW (Li et al., 2021b; Y. R. Li et al., 2022; Zhu et al., 2019). The Lenti/iNKT-sr39TK vector was constructed by inserting into pMNDW vector a synthetic tricistronic gene encoding human iNKT TCRα-F2A-TCRβ-P2A-sr39TK; the Lenti/FG vector was constructed by inserting into pMNDW a synthetic bicistronic gene encoding Fluc-P2A-EGFP. The synthetic gene fragments were obtained from GenScript and IDT. Lentiviruses were produced using HEK 293T cells, following a standard calcium precipitation protocol and an ultracentrifigation concentration protocol (Li et al., 2021b; Y. R. Li et al., 2022; Zhu et al., 2019). Lentivector titers were measured by transducing HT29 cells with serial dilutions and performing digital qPCR (Li et al., 2021b; Y. R. Li et al., 2022; Zhu et al., 2019).

To make stable tumor cell lines overexpressing firefly luciferase and enhanced green fluorescence protein (FG) dual-reporters, parental tumor cell lines were transduced with lentiviral vectors encoding the intended gene(s). 72 h following lentiviral transduction, cells were subjected to flow cytometry sorting to isolate gene-engineered cells for making stable cell lines. Two stable tumor cell lines were generated for this study, including Raji-FG and HL60-FG.

### Human Periphery Blood Mononuclear Cells (PBMCs)

Healthy donor human PBMCs were obtained from the UCLA/CFAR Virology Core Laboratory, with identification information removed under federal and state regulations. Cells were cryopreserved in Cryostor CS10 (BioLife Solution) using CoolCell (BioCision) and were stored in liquid nitrogen for all experiments and long-term storage.

### Media and Reagents

α-Galactosylceramide (αGC, KRN7000) was purchased from Avanti Polar Lipids. Recombinant human IL-2, IL-3, IL-4, IL-7, IL-15, Flt3-Ligand, Stem Cell Factor (SCF), Thrombopoietin (TPO), and Granulocyte-Macrophage Colony-Stimulating Factor (GM-CSF) were purchased from Peprotech. Ganciclovir (GCV) was purchased from Sigma.

X-VIVO 15 Serum-Free Hematopoietic Cell Medium was purchased from Lonza. RPMI 1640 and DMEM cell culture medium were purchased from Coming Cellgro. Fetal bovine serum (FBS) was purchased from Sigma. Medium supplements, including Penicillin-Streptomycine-Glutamine (P/S/G), MEM non-essential amino acids (NEAA), HEPES Buffer Solution, and Sodium Pyruvate, were purchased from GIBCO. Beta-Mercaptoethanol (R-ME) was purchased from Sigma. Normocin was purchased from InvivoGen. Complete lymphocyte culture medium (denoted as C10 medium) was made of RPMI 1640 supplemented with FBS (10% vol/vol), P/S/G (1% vol/vol), MEM NEAA (1% vol/vol), HEPES (10 mM), Sodium Pyruvate (1 mM), (3-ME (50 mM), and Normocin (100 mg/ml). Medium for culturing human Raji and HL60 tumor cell lines (denoted as R10 medium) was made of RPMI 1640 supplemented with FBS (10% vol/vol) and P/S/G (1% vol/vol). Medium for culturing HEK 293T cell line (denoted as D10 medium) was made of DMEM supplemented with FBS (10% vol/vol) and P/S/G (1% vol/vol).

### Method Details

### Antibodies and Flow Cytometry

All flow cytometry stains were performed in PBS for 15 min at 4° C. The samples were stained with Fixable Viability Dye eFluor506 (e506) mixed with Mouse Fc Block (anti-mouse CD16/32) or Human Fc Receptor Blocking Solution (TrueStain FcX) prior to antibody staining. Antibody staining was performed at a dilution according to the manufacturer's instructions. Fluorochrome-conjugated antibodies specific for human CD45 (Clone H130), TCRαβ (Clone I26), CD4 (Clone OKT4), CD8 (Clone SKI), CD45RO (Clone UCHL1), CD161 (Clone HP-3G10), CD69 (Clone FN50), CD56 (Clone HCD56), CD62L (Clone DREG-56), CD14 (Clone HCD14), CD1d (Clone 51.1), NKG2D (Clone 1D11), DNAM-1 (Clone 11A8), IFN-γ (Clone B27), Granzyme B (Clone QA16A02), Perforin (Clone dG9), TNF-β (Clone Mab11), IL-2 (Clone MQ1-17H12), HLA-A2 (Clone BB7.2) were purchased from BioLegend; Fluorochrome-conjugated antibodies specific for human CD34 (Clone 581) and TCR Vα24-Jβ18 (Clone 6B11) were purchased from BD Biosciences. Human Fc Receptor Blocking Solution (TrueStain FcX) was purchased from Biolegend, and Mouse Fc Block (anti-mouse CD16/32) was purchased from BD Biosciences. Fixable Viability Dye e506 were purchased from Affymetrix eBioscience. Intracellular cytokines were stained using a Cell Fixation/Permeabilization Kit (BD Biosciences). Stained cells were analyzed using a MACSQuant Analyzer 10 flow cytometer (Miltenyi Biotech). FlowJo software was utilized to analyze the data.

### Enzyme-Linked Immunosorbent Cytokine Assays (ELISAs)

The ELISAs for detecting human cytokines were performed following a standard protocol from BD Biosciences. Supernatants from co-culture assays were collected and assayed to quantify IFN-γ. Capture and biotinylated pairs for detecting cytokines were purchased from BD Biosciences. The streptavidin-HRP conjugate was purchased from Invitrogen. Human cytokine standards were purchased from eBioscience. Tetramethylbenzidine (TMB) substrate was purchased from KPL. The samples were analyzed for absorbance at 450 nm using an Infinite M1000 microplate reader (Tecan).

In Vitro Generation of HSC-Engineered iNKT (HSC-iNKT) Cells

Cord blood-derived human CD34⁺ hematopoietic stem and progenitor cells (denoted as HSCs) were obtained from HemaCare. Frozen-thawed HSCs were revived in HSC-culture medium comprised of X-VIVO 15 Serum-Free Hematopoietic Cell Medium supplemented with human recombinant SCF (50 ng/ml), FLT3-L (50 ng/ml), TPO (50 ng/ml), and IL-3 (10 ng/ml) for 24 hours. Cells were then transduced with Lenti/iNKT-sr39TK viruses for another 24 hours (Li et al., 2021b; Y. R. Li et al., 2022; Zhu et al., 2019). The transduced HSCs were then collected and put into an Artificial Thymic Organoid (ATO) culture or a Feeder-Free culture.

In the ATO culture, transduced HSCs were mixed with MS5-DLL4 feeder cells to form ATOs and cultured over ˜8 weeks following a previously established protocol (Li et al., 2021b; Montel-Hagen et al., 2019). In the Feeder-Free culture, transduced HSCs were cultured using a StemSpan™ T Cell Generation Kit (StemCell Technologies) over ˜5 weeks following the manufacturer's instructions (Y. R. Li et al., 2022). The resulting HSC-iNKT cells isolated from ATOs or Feeder-Free culture were expanded with αGC-loaded PBMCs (αGC-PBMCs). To prepare αGC-PBMCs, 1-10×10⁷ PBMCs were incubated in 5 ml Cl 0 medium containing 5 μg/ml αGC for 1 hour, followed by irradiation at 6,000 rads. HSC-iNKT cells were mixed with irradiated αGC-PBMCs at ratio 1:1, followed by culturing for 2 weeks in C10 medium supplemented with human IL-7 (10 ng/ml) and IL-15 (10 ng/ml); cell cultures were split, and fresh media/cytokines were added if needed. The resulting HSC-iNKT cell products were then collected and cryopreserved for future use.

Generation of PBMC-Derived Conventional T (PBMC-Tcon) and iNKT (PBMC-iNKT) Cells

Healthy donor PBMCs were obtained from the UCLA/CFAR Virology Core Laboratory and were used to generate the PBMC-Tc and PBMC-iNKT cells.

To generate PBMC-Tcon cells, PBMCs were stimulated with CD3/CD28 T-activator beads (ThermoFisher Scientific) and cultured in C10 medium supplemented with human IL-2 (20 ng/mL) for 2-3 weeks, following the manufacturer's instructions.

To generate PBMC-iNKT cells, PBMCs were enrich for iNKT cells using anti-iNKT microbeads (Miltenyi Biotech) and MACS-sorting, followed by stimulation with donor-matched irradiated αGC-PBMCs at the ratio of 1:1 and cultured in C10 medium supplemented with human recombinnat IL-7 (10 ng/ml) and IL-15 (10 ng/ml) for 2-3 weeks. If necessary, the resulting PBMC-iNKT cells could be further purified using Fluorescence-Activated Cell Sorting (FACS) via human iNKT TCR antibody (Clone 6B11; BD Biosciences) staining.

### HSC-iNKT Cell Phenotype and Functional Study

HSC-iNKT cells were analyzed in comparison with PBMC-Tcon and PBMC-iNKT cells. Phenotype of these cells was studied using flow cytometry by analyzing cell surface markers including co-receptors (i.e., CD4 and CD8), NK cell receptors (i.e., CD161, NKG2D, and DNAM-1), and memory T cell markers (i.e., CD45RO). The capacity of these cells to produce cytokines (i.e., IFN-γ, TNF-α, IL-2, and IL-4) and cytotoxic molecules (i.e., Perform and Granzyme B) were studied using flow cytometry via intracellular staining.

### Ganciclovir (GCV) In Vitro and In Vivo Killing Assay

For GCV in vitro killing assays, HSC-iNKT cells were cultured in C10 medium in the presence of titrated amount of GCV (0-50 μM) for 4 days; live HSC-iNKT cells were then counted using a hematocytometer (VWR) via Trypan Blue staining (Fisher Scientific).

GCV in vivo killing assay was performed using an NSG xenograft mouse model. NSG mice received i.v. injection of 1×10⁷ HSC-iNKT cells on day 0, followed by i.p. injection of GCV for 5 consecutive days (50 mg/kg per injection per day). On day 5, mice were terminated. Multiple tissues (i.e., blood, spleen, liver, and lung) were collected and processed for flow cytometry analysis to detect tissue-infiltrating HSC-iNKT cells (identified as iNKT TCR⁺CD45⁺), following established protocols (Li et al., 2021b; Y. R. Li et al., 2022; Zhu et al., 2019).

### In Vitro Tumor Cell Killing Assay

Tumor cells (1×10⁴ cells per well) were co-cultured with HSC-iNKT cells (at ratios indicated in figure legends) in Coming 96-well clear bottom black plates for 24 hours, in C10 medium. At the end of culture, live tumor cells were quantified by adding D-luciferin (150 μg/ml; Caliper Life Science) to cell cultures and reading out luciferase activities using an Infinite M1000 microplate reader (Tecan).

In some experiments, 10 μg/ml of LEAF™ purified anti-human NKG2D (Clone 1D11, Biolegend), anti-human DNAM-1 antibody (Clone 11A8, Biolegend), or LEAF™ purified mouse 1gG2bk isotype control antibody (Clone MG2B-57, Biolegend) was added to co-cultures, to study NK activating receptor-mediated tumor cell killing mechanism.

In Vitro Mixed Lymphocyte Reaction (MLR) Assay: Studying ^{3rd}HSC-iNKT Cell Inhibition of Allogeneic T Cell Response

PBMCs of multiple healthy donors were irradiated at 2,500 rads and used as stimulators, and non-irradiated allogeneic PBMCs were used as responders. In order to separate the different donor PBMCs when performing flow cytometry, HLA-A2⁺ responders and HLA-A2⁻ stimulators were used in this study. Irradiated stimulators (2.5×10⁵ cells/well) and responders (1×10⁴ cells/well) were co-cultured with or without the addition of ^{3rd}HSC-iNKT cells (1×10⁴ cells/well) in 96-well round bottom plates in C10 medium for up to 4 days. For detection of composition and phenotype using flow cytometry, cells were collected on day 1. For IFN-γ production using ELISA, cell culture supernatants were collected on day 4. To study CD1d-dependent killing mechanism of ^{3rd}HSC-iNKT cells, 10 g/ml of LEAF™ purified anti-human CD1d (Clone 51.1, Biolegend) or LEAF™ purified mouse lgG2bk isotype control antibody (Clone MG2B-57, Biolegend) was added to co-cultures.

### Bioluminescence Live Animal Imaging (BLI)

BLI was performed using a Spectral Advanced Molecular Imaging (AMI) HTX imaging system (Spectral instrument Imaging). Live animal imaging was acquired 5 minutes after intraperitoneal (i.p.) injection of D-Luciferin (1 mg per mouse). Imaging results were analyzed using an AURA imaging software (Spectral Instrument Imaging).

Human PBMC Xenograft NSG Mouse Model: Studying ^{3rd}HSC-iNKT Cell Amelioration of GvHD

NSG mice were pre-conditioned with 100 rads of total body irradiation (day −1), followed by intravenous injection of 2×10⁷ healthy donor PBMCs with or without the addition of 2×10^{7 3rd}HSC-iNKT cells. Mice were weighed daily, bled weekly, and scored 0-2 per clinical sign of GvHD (i.e., body weight, activity, posture, skin thickening, diarrhea, and dishevelment). Mice were terminated and analyzed when moribund. Various mouse tissues (i.e., blood, spleen, liver, lung, bone marrow, skin, and salivary ligand) were harvested and processed for either flow cytometry or histologic analysis.

### Human PBMC Xenograft NSG Mouse Model: Studying CD14′ Myeloid Cell Modulation of GvHD

NSG mice were pre-conditioned with 100 rads of total body irradiation (day −1), followed by intravenous injection of 2×10⁷ healthy donor PBMCs or 9×10⁶ CD14-depleted donor PBMCs. The amount of PBMCs given was normalized to contain the same number of T cells. Mice were weighed daily, bled weekly, and scored 0-2 per clinical sign of GvHD (i.e., body weight, activity, posture, skin thickening, diarrhea, and dishevelment).

Human CD14-Depleted PBMC Xenograft NSG Mouse Model: Studying ^{3rd}HSC-iNKT Cell Amelioration of GvHD

NSG mice were pre-conditioned with 100 rads of total body irradiation (day −1), followed by intravenous injection of 9×10⁶ CD14-depleted donor PBMCs with or without the addition of 2×10^{7 3rd}HSC-iNKT cells. Mice were weighed daily, bled weekly, and scored 0-2 per clinical sign of GvHD (i.e., body weight, activity, posture, skin thickening, diarrhea, and dishevelment). Mice were terminated and analyzed when moribund.

Raji-FG Human B Cell Lymphoma Xenograft NSG Mouse Model: Studying ^{3rd}HSC-iNKT Cell Retention of GvL Effect

NSG mice were pre-conditioned with 100 rads of total body irradiation (day −1), followed by subcutaneous inoculation with 1×10⁵ Raji-FG cells (day 0). On day 3, the tumor-bearing experimental mice received intravenous (i.v.) injection of 2×10⁷ healthy donor PBMCs with or without the addition of 2×10^{7 3rd}HSC-iNKT cells. Tumor load were monitored over time using BLI. Mice were also weighed daily, bled weekly, and scored 0-2 per clinical sign of GvHD (i.e., body weight, activity, posture, skin thickening, diarrhea, and dishevelment). Mice were terminated and analyzed when moribund.

HL60-FG Human Acute Myeloid Leukemia Xenograft NSG Mouse Model: Studying ^{3rd}HSC-iNKT Cell Retention of GvL Effect

NSG mice were pre-conditioned with 175 rads of total body irradiation (day −1), followed by intravenous inoculation with 2×10⁵ HL60-FG (day 0). On day 3, the tumor-bearing experimental mice received intravenous (i.v.) injection of 2×10⁷ healthy donor PBMCs with or without the addition of 2×10^{7 3rd}HSC-iNKT cells. Tumor load were monitored over time using BLI. Mice were also weighed daily, bled weekly, and scored 0-2 per clinical sign of GvHD (i.e., body weight, activity, posture, skin thickening, diarrhea, and dishevelment). Mice were terminated and analyzed when moribund.

### Histological Analysis

Tissues (i.e., liver, lung, salivary glands, and skin) were collected from the experimental mice, fixed in 10% Neutral Buffered Formalin for up to 36 hours, then embedded in paraffin for sectioning (5 μm thickness). Tissue sections were prepared and stained with Hematoxylin and Eosin (H&E) or anti-CD3 by the UCLA Translational Pathology Core Laboratory, following the Core's standard protocols. The H&E-stained sections were imaged on a Zeiss Observer II upright microscope. All images were captured at either 100× or 200× and processed using Zen Blue software. GvHD pathological score was calculated as follows: skin: epidermal changes (0-3), dermal changes (0-3), adipose changes (0-3); salivary: infiltration (0-4), follicular destruction (0-4); liver: duct infiltration (0-3), number of ducts involved (0-3), liver cell apoptosis (0-3); lung: infiltrates (0-3); pneumonitis (0-3), overall appearance (0-3). For CD3 surface area measurements, the anti-CD3-stained sections were scanned in their entirety using Hamamatsu Nanozoomer 2.0 HT. The % CD3⁺ area was determined by CD3⁺ area divided by total tissue area, using an Image-Pro Premier software.

### Statistical Analysis

GraphPad Prism 6 (Graphpad Software) was used for statistical data analysis. Student's two-tailed t test was used for pairwise comparisons. Ordinary 1-way ANOVA followed by Tukey's multiple comparisons test was used for multiple comparisons. Log rank (Mantel-Cox) test adjusted for multiple comparisons was used for Meier survival curves analysis. Data are presented as the mean±SEM, unless otherwise indicated. In all figures and figure legends, “N” represents the number of samples or animals utilized in the indicated experiments. A P value of less than 0.05 was considered significant. ns, not significant; *P<0.05; **P<0.01; ***P<0.001; ****P<0.0001.

### Discussion

iNKT cells are uniquely positioned at the crossroads of innate and adaptive immunity and have potent immunoregulatory functions in a variety of diseases (Brennan et al., 2013; Van Kaer et al., 2011). Research into harnessing iNKT cells to combat GvHD began decades ago (Lan et al., 2001), but clinical application of iNKT cells has been hindered by their scarcity in peripheral blood (Krijgsman et al., 2018). We have recently developed an ex vivo HSC-iNKT culture method that can robustly generate large quantities of pure, clonal human iNKT cells (FIGS. 1 and S1 in Li et al.) (Li et al., 2021b; Y. R. Li et al., 2022). The resulting third-party HSC-iNKT (^{3rd}HSC-iNKT) cells closely resembled peripheral blood-derived endogenous CD4-iNKT cells and displayed anti-GvHD activity while preserving GvL effects in preclinical models of leukemia and lymphoma (FIGS. 2-6 and S2-S5 in Li et al.). Importantly, such ^{3rd}HSC-iNKT cells do not cause GvHD themselves and are resistant to allorejection due to their intrinsic low expression of HLA-I and II molecules (Li et al., 2021b; Y. R. Li et al., 2022), highlighting their potential for off-the-shelf anti-GvHD therapy.

GvHD prophylaxis is centered around calcineurin inhibitor (CNT)-based therapy and investigations into new methods, including those depleting T cells, modulating T cell co-stimulatory pathways (e.g., checkpoints), enhancing regulatory T cells, targeting T cell trafficking, and altering cytokine pathways (Gooptu and Antin, 2021). Despite prophylactic interventions, acute GvHD is a common complication of allo-HSCT, occurring in 30-50% of patients, 14-36% of whom develop severe acute GvHD, and is a major cause of morbidity and mortality (Malard et al., 2020). The current first-line treatment for acute GvHD is systemic steroid therapy, but almost half of patients will become refractory to treatment and there is no accepted standard-of-care treatment for steroid refractory-acute GvHD (Malard et al., 2020). The dismal survival rate and poor quality of life in these patients highlight the urgent need for novel therapeutic and prophylactic agents against acute GvHD.

The driver of clinical acute GvHD is donor alloreactive T cells (Ball and Egeler, 2008). Following lymphodepletion and HSCT, host and donor antigen-presenting cells respond to host tissue damage and lead to the activation of donor T cells (Ramachandran et al., 2019). Although culpable for GvHD, HSCT-derived T cells are essential for antitumor effects, as their depletion from HSCT grafts precipitates increased relapse rates (Horowitz et al., 1990). To study the anti-GvHD potential of ^{3rd}HSC-iNKT cells, we adopted an xeno-GvHD NSG mouse model, in which human PBMCs are intravenously infused and subsequent donor T cell activation results in GvHD (FIGS. 2 and S2 in Li et al.), replicating some of the components of clinical GvHD (Ali et al., 2012; King et al., 2009).

Although mechanisms are currently under investigation, our ex vivo culture of iNKT TCR transduced HSCs produces nearly all CD4⁻ HSC-iNKT cells (FIGS. 1B, 1C and 1E) (Li et al., 2021b). Like PBMC-derived endogenous CD4⁻ iNKT cells, the engineered HSC-iNKT cells express large amounts of IFN-γ and TNF-α as well as Granzyme B and Perform (FIG. 1) (Li et al., 2021b), indicative of a Th1 cytokine profile and cytotoxic potential (Li et al., 2021b, 2021a). Additionally, these HSC-iNKT cells show low response to IL-12/IL-18 innate signaling in vitro (Data not shown). In 2012, Chaidos and colleagues conducted a comprehensive analysis of all immune populations in allogeneic HSCT grafts, and found that only CD4⁻ iNKT cells were correlated with reduced acute GvHD occurrence (Chaidos et al., 2012). Five years later, Rubio and colleagues also revealed that only pre-transplant donor CD4⁻ iNKT cells predicted clinical acute GvHD following HSCT (Rubio et al., 2017). Corroborating the clinical findings, a preclinical study from the same research team confirmed CD4⁻ iNKT cells, but not CD4⁺ iNKT cells, prevented GvHD using a xenograft NSG mouse model (Coman et al, 2018), and in vitro assays revealed that CD4⁻ iNKT cells reduced the maturation and induced the apoptosis of human DCs (Coman et al., 2018). In our study, ^{3rd}HSC-iNKT cells ameliorated GvHD though depleting donor CD14⁺ myeloid cells, at least partly via CD1d recognition (FIGS. 3 and 4). Interestingly, CD4⁺ subpopulation of iNKT cells has also been implicated in GvHD amelioration, albeit through different mechanisms (Chaidos et al., 2012; Coman et al., 2018; Mavers et al., 2017; Rubio et al., 2012). The beneficial role in GvHD has been attributed to IL-4-induced Treg expansion in preclinical syngeneic mouse models (Lan et al., 2003; Pillai et al., 2007; Schneidawind et al., 2015, 2014). It would be an interesting future direction to modify our ex vivo HSC-iNKT cell culture to produce CD4⁺ human iNKT cells to harness the anti-GvHD potential of this subpopulation of iNKT cells.

iNKT cells can also play a direct role in tumor killing. Through CD1d dependent and independent means, iNKT cells have been shown to lysis a variety of tumor cells (King et al., 2018; Li et al., 2021b; Zhu et al., 2019). Furthermore, in hematological and solid tumor models, adoptive transfer of iNKT cells reduces tumor burden and enhances overall survival (Fujii et al., 2013). Our previous studies have demonstrated the antitumor functions of HSC-iNKT cells in vivo when targeting CD1d positive and negative cancer cells (Li et al., 2021b; Zhou et al., 2021). Importantly, HSC-iNKT cells do not recognize mismatched MHCs and thus pose no risk of inducing GvHD; furthermore, due to their intrinsic low expression of HLA-I and II molecules, these cells are resistant to allorejection (Li et al., 2021b; Y. R. Li et al., 2022). These features of HSC-iNKT cells make them suitable for allogeneic cell therapy.

Allo-HSCT is an established, effective treatment for hematological malignancies, but GvHD is common and debilitating adverse event for many allo-HSCT recipients. We propose to develop the off-the-shelf HSC-iNKT cell therapy to ameliorate GvHD while preserving GvL in the treatment of blood cancers. The reported ex vivo HSC-iNKT cell culture is robust and of high yield and purity, with the potential of being scaled for further translation and clinical development. From one cord blood donor, over 10,000 doses of third-party HSC-iNKT cells can be manufactured and cryopreserved for ready distribution to allo-HSCT patients; MHC matching is not needed. This study highlights the potential of ^{3rd}HSC-iNKT cells to address a critical unmet medical need and warrants further investigations of this promising off-the-shelf cell product.

### Limitations of the Study

Predominant mouse models studying GvHD typically employ transplantation of T cell-depleted bone marrow and donor-derived T cells into lethally irradiated recipients; these are paramount to advance the forefront of knowledge regarding the incidence of GvHD within allo-HSCT therapeutics (Schroeder and DiPersio, 2011). In this study, healthy donor T cells were used to generate a PBMC-xenograft NSG mouse model, producing a construct where T cell-mediated GvHD could be studied and manipulated in vivo. However, limitations to this model preclude its ability to fully reflect GvHD pathology in allo-HSCT. Such complexity arises from factors such as the restricted availability of human embryonic tissue for transplant, the need for sublethal total body irradiation, demand for a high quantity of human PBMCs, and instability in the onset window of GvHD (Huang et al., 2018). Additionally, murine immunoreaction after engraftment of human immune cells is highly distinct compared to that in humans in regard to both biological phenotype and genetics. Therefore, developing a model that more accurately mimics human GvHD pathology, while reducing variance from these limitations, is necessary to understand patient reactivity to allo-HSCT therapies.

### Star Methods

Detailed methods are provided in the online version of this paper and include the following:
KEY RESOURCES TABLE
RESOURCE AVAILABILITY
   Lead Contact
   Materials Availability
   Data and Code Availability
EXPERIMENTAL MODEL AND SUBJECT DETAILS
   Mice
   Cell Lines and Viral Vectors
   Human Periphery Blood Mononuclear Cells (PBMCs)
   Media and Reagents
METHOD DETAILS
   Antibodies and Flow Cytometry
   Enzyme-Linked Immunosorbent Cytokine Assays (ELISAs)
   In Vitro Generation of HSC-Engineered iNKT (HSC-iNKT) Cells
   Generation of PBMC-Derived Conventional T (PBMC-Tcon) and iNKT (PBMC-iNKT) Cells
   HSC-iNKT Cell Phenotype and Functional Study
   Ganciclovir (GCV)In Vitro and In Vivo Killing Assay
   In Vitro Tumor Cell Killing Assay
   In Vitro Mixed Lymphocyte Reaction (MLR) Assay: Studying ^{3rd}HSC-iNKT Cell Inhibition of Allogeneic T cell Response
   Bioluminescence Live Animal Imaging (BLI)
   Human PBMC Xenograft NSG Mouse Model: Studying ^{3rd}HSC-iNKT Cell Amelioration of GvHD
   Human PBMC Xenograft NSG Mouse Model: Studying CD14⁺ Myeloid Cell Modulation of GvHD
   Human CD14-Depleted PBMC Xenograft NSG Mouse Model: Studying ^{3rd}HSC-iNKT Cell Amelioration of GvHD
   Raji-FG Human B Cell Lymphoma Xenograft NSG Mouse Model: Studying ^{3rd}HSC-iNKT Cell Retention of GvL Effect
   HL60-FG Human Acute Myeloid Leukemia Xenograft NSG Mouse Model: Studying ^{3rd}HSC-iNKT Cell Retention of GvL Effect
   Histological Analysis
   Statistical Analysis

### Star*Methods

KEY RESOURCES TABLE

| | | |
|---|---|---|
| REAGENT or RESOURCE | SOURCE | IDENTIFIER |

Antibodies

| | | |
|---|---|---|
| Anti-human IFN-γ (ELISA, capture) | BD Biosciences | CAT#551221, |
| | | RRID: AB_394099 |
| Anti-human IFN-γ (ELISA, detection) | BD Biosciences | CAT#554550, |
| | | RRID: AB_395472 |
| Anti-human CD34 (Clone 581) | BD Biosciences | CAT#555822, |
| | | RRID: AB_396151 |
| Anti-human TCR Vα24-Jβ18 (Clone 6B11) | BD Biosciences | CAT#552825, |
| | | RRID: AB_394478 |
| Anti-human CD45 (Clone H130) | Biolegend | CAT#304026, |
| | | RFID: AB_893337 |
| Anti-human TCRαβ (Clone 126) | Biolegend | CAT#306716, |
| | | RRID: |
| | | AB_1953257 |
| Anti-human CD4 (Clone OKT4) | Biolegend | CAT#317414, |
| | | RRID: AB_571959 |
| Anti-human CD8 (Clone SK1) | Biolegend | CAT#344714, |
| | | RRID: |
| | | AB_2044006 |
| Anti-human CD45RO (Clone UCHL1) | Biolegend | CAT#304216, |
| | | RRID: AB_493659 |
| Anti-human CD161 (Clone HP-3G10) | Biolegend | CAT#339928, |
| | | RRID: |
| | | AB_2563967 |
| Anti-human CD14 (Clone HCD14) | Biolegend | CAT#325608, |
| | | RRID: AB_830681 |
| Anti-human CD19 (Clone SJ25C1) | Biolegend | CAT#363005, |
| | | RRID: |
| | | AB_2564127 |
| Anti-human CD11b (Clone ICRF44) | Biolegend | CAT#301330, |
| | | RRID: |
| | | AB_2561703 |
| Anti-human CD1d (Clone 51.1) | Biolegend | CAT#350308, |
| | | RRID: |
| | | AB_10642829 |
| Anti-human NKG2D (Clone 1D11) | Biolegend | CAT#320812, |
| | | RRID: |
| | | AB_2234394 |
| Anti-human DNAM-1 (Clone 11A8) | Biolegend | CAT#338312, |
| | | RRID: |
| | | AB_2561952 |
| Anti-human HLA-A2 (Clone BB7.2) | Biolegend | CAT#343308, |
| | | RRID: |
| | | AB_2561567 |
| Anti-human IFN-γ (Clone B27) | Biolegend | CAT#506518, |
| | | RRID: |
| | | AB_2123321 |
| Anti-human Granzyme B (Clone QA16A02) | Biolegend | CAT#372204, |
| | | RRID: |
| | | AB_2687028 |
| Anti-human Perforin (Clone dG9) | Biolegend | CAT#308126, |
| | | RRID: |
| | | AB_2572049 |
| Anti-human TNFα (Clone Mab11) | Biolegend | CAT#502912, |
| | | RRID: AB_315264 |
| Anti-human IL-2 (Clone MQ1-17H12) | Biolegend | CAT#500341, |
| | | RRID: |
| | | AB_2562854 |
| Anti-human IL-4 (Clone MP4-25D2) | Biolegend | CAT#500824, |
| | | RRID: |
| | | AB_2126746 |
| Anti-human GM-CSF (Clone BVD2-21C11) | Biolegend | CAT#502313, |
| | | RRID: |
| | | AB_2561838 |
| LEAF purified anti-human NKG2D antibody | Biolegend | CAT#320810, |
| (Clone 1D11) | | RRID: |
| | | AB_2133276 |
| LEAF purified anti-human DNAM-1 antibody | BD Biosciences | CAT#559786, |
| (Clone DX11) | | RRID: AB_397327 |
| Mouse IgG1, κ isotype control antibody | Biolegend | CAT#400124, |
| (Clone MOPC-21) | | RRID: |
| | | AB_2890215 |
| LEAF purified anti-human CD1d antibody | Biolegend | CAT#350304, |
| (Clone 51.1) | | RRID: |
| | | AB_10641291 |
| LEAF purified Mouse IgG2b, k isotype ctrl | Biolegend | CAT#401201, |
| (Clone MG2b-57) | | RRID: |
| | | AB_2744505 |
| Human Fc Receptor Blocking Solution | Biolegend | CAT#422302, |
| (TrueStain FcX) | | RRID: |
| | | AB_2818986 |
| Mouse Fc Block (anti-mouse CD16/32) | BD Biosciences | CAT#553142, |
| | | RRID: AB_394657 |

Bacterial and virus strains

| | | |
|---|---|---|
| Lenti/iNKT-sr39TK | This paper | N/A |
| Lenti/FG | This paper | N/A |

Biological samples

| | | |
|---|---|---|
| Human peripheral blood mononuclear cells | UCLA | N/A |
| (PBMCs) | | |
| Cord Blood Cryo CD34 | HemaCare | CAT#CB34C-3 |

Chemicals, peptides, and recombinant proteins

| | | |
|---|---|---|
| Streptavidin-HRP conjugate | Invitrogen | CAT#SA10001 |
| IFN-γ (ELISA, standard) | eBioscience | CAT#29-8319-65 |
| Tetramethylbenzidine (TMB) | KPL | CAT#5120-0053 |
| Ganciclovir (GCV) | Sigma | CAT#ADV465749843 |
| Recombinant human IL-2 | Peprotech | CAT#200-02 |
| Recombinant human IL-3 | Peprotech | CAT#200-03 |
| Recombinant human IL-7 | Peprotech | CAT#200-07 |
| Recombinant human IL-15 | Peprotech | CAT#200-15 |
| Recombinant human Flt3-Ligand | Peprotech | CAT#300-19 |
| Recombinant human SCF | Peprotech | CAT#300-07 |
| Recombinant human TPO | Peprotech | CAT#300-18 |
| Recombinant human GM-CSF | Peprotech | CAT#300-03 |
| L-ascorbic acid 2-phosphate | Sigma | CAT#A8960-5G |
| B27 ™ Supplement (50X), serum free | ThermoFisher | CAT#17504044 |
| α-Galactosylceramide (KRN7000) | Avanti Polar | SKU#867000P- |
| | Lipids | 1 mg |
| X-VIVO 15 Serum-free Hematopoietic Cell | Lonza | CAT#04-418Q |
| Medium | | |
| RPMI1640 cell culture medium | Corning Cellgro | CAT#10-040-CV |
| DMEM cell culture medium | Corning Cellgro | CAT#10-013-CV |
| Fetal Bovine Serum (FBS) | Sigma | CAT#F2442 |
| MACS BSA stock solution | Miltenyi | CAT#130-091-376 |
| 30% BSA | Gemini | CAT#50-753-3079 |
| Penicillin-Streptomycine-Glutamine (P/S/G) | Gibco | CAT#10378016 |
| Penicillin:streptomycin (pen:strep) | Gemini | CAT#400-109 |
| solution (P/S) | Bio-products | |
| MEM non-essential amino acids (NEAA) | Gibco | CAT#11140050 |
| HEPES Buffer Solution | Gibco | CAT#15630056 |
| Sodium Pyruvate | Gibco | CAT#11360070 |
| Beta-Mercaptoethanol | Sigma | SKU#M6250 |
| Normocin | InvivoGen | CAT#ant-nr-2 |
| Cell Fixation/Permeabilization Kit | BD Biosciences | CAT#554714 |
| RetroNectin recombination human fibronectin | Takara | CAT#T100B |
| fragment, 2.5 mg | | |
| 10% neutral-buffered formalin | Richard-Allan | CAT#5705 |
| | Scientific | |
| D-Luciferin | Caliper LIfe | CAT#XR-1001 |
| | Science | |
| Isoflurane | Zoetis | CAT#50019100 |
| Phosphate Buffered Saline (PBS) pH 7.4 (1X) | Gibco | CAT#10010-023 |
| Formaldehyde | Sigma-Aldrich | CAT#F8775 |
| Golgistop Protein Transport Inhibitor | BD Biosciences | CAT#554724 |
| Phorbol-12-myristate-13-acetate (PMA) | Calbiochem | CAT#524400 |
| Ionomycin, Calcium salt, | Calbiochem | CAT#407952 |
| Streptomyces conglobatus | | |
| Poloxamer Synperonic F108 | Sigma | CAT#07579-250G- |
| | | F |
| Prostaglandin E2 | Cayman Chemical | CAT#14-190-136 |
| Fixable Viability Dye eFluor506 | affymetrix | CAT#65-0866-14 |
| | eBioscience | |

Critical commercial assays

| | | |
|---|---|---|
| Human CD34 MicroBeads Kit | Miltenyi Biotec | CAT#130-046-703 |
| Human CD14 MicroBeads Kit | Miltenyi Biotec | CAT#130-050-201 |
| Human Anti-iNKT MicroBeads | Miltenyi Biotec | CAT#130-094-842 |
| Fixation/Permeabilization Solution Kit | BD Sciences | CAT#55474 |
| StemSpan ™ Lymphoid Differentiation Coating | Stem Cell | CAT#9925 |
| Material (100X) | Technologies | |
| StemSpan ™ SFEM II | Stem Cell | CAT#9605 |
| | Technologies | |
| ImmunoCult ™ Human CD3/CD28/CD2 T Cell | Stem Cell | CAT#10970 |
| Activator | Technologies | |
| Cryostor cell cryopreservation media | Sigma | CAT#C2874- |
| | | 100 ML |

Experimental models: Cell lines

| | | |
|---|---|---|
| Human Burkitts lymphoma cell line Raji | ATCC | CCL-86 |
| Human acute myeloid leukemia cell line HL60 | ATCC | CCL-240 |
| Human Burkitts lymphoma cell line Raji-FG | This paper | N/A |
| Human acute myeloid leukemia cell | This paper | N/A |
| line HL60-FG | | |

Experimental models: Organisms/strains

| | | |
|---|---|---|
| NOD.Cg-Prkdcscid Il2rgtm1Wjl/SzJ (NSG) | The Jackson | Stock #: 005557 |
| | Laboratory | |

Recombinant DNA

| | | |
|---|---|---|
| Vector: parental lentivector pMNDW | Giannoni et | N/A |
| | al., 2013 | |
| | and Lan et | |
| | al., 2006 | |

Software and algorithms

| | | |
|---|---|---|
| FlowJo Software | FlowJo | https://www.flowjo.com/solutions/flowjo/downloads |
| Living Imaging 2.50 software | Xenogen/PerkinElmer | http://www.perkinelmer.com/lab- |
| | | products-and-services/resources/in- |
| | | vivo-imaging-software-downloads.html |
| AURA imaging software | Spectral | https://spectralinvivo.com/software/ |
| | Instruments | |
| | Imaging | |
| I-control 1.7 Microplate Reader Software | Tecan | https://www.selectscience.net/tecan/i- |
| | | control-microplate-reader-software/81307 |
| ImageJ | ImageJ | https://imagej.net/Downloads |
| Prism 6 | Graphpad | https://www.graphpad.com/scientific- |
| | | software/prism/ |

### Keywords

Invariant natural killer T (iNKT) cells; Hematopoietic stem cell (HSC) engineering; Ex vivo T cell differentiation; Graft versus host disease (GvHD); Graft versus leukemia/lymphoma (GvL) effect

### Supplemental Information

Supplemental Information discussed herein (e.g., FIG. S1A) is found in Li et al. which is incorporated herein by reference.

### REFERENCES

Ali, N., Flutter, B., Sanchez Rodriguez, R., Sharif-Paghaleh, E., Barber, L. D., Lombardi, G., Nestle, F. O., 2012. Xenogeneic Graft-versus-Host-Disease in NOD-scid IL-2Rynull Mice Display a T-Effector Memory Phenotype. PLoS One 7, 1-10. https://doi.org/10.1371/journal.pone.0044219
Anderson, B. E., McNiff, J. M., Jain, D., Blazar, B. R., Shlomchik, W. D., Shlomchik, M. J., 2005. Distinct roles for donor- and host-derived antigen-presenting cells and costimulatory molecules in murine chronic graft-versus-host disease: requirements depend on target organ. Blood 105, 2227-2234. https://doi.org/10.1182/blood-2004-08-3032
Appelbaum, F. R., 2001. Haematopoietic cell transplantation as immunotherapy. Nature 411, 385-389. https://doi.org/10.1038/35077251
Apperley, J. F., Jones, L., Hale, G., Waldmann, H., Hows, J., Rombos, Y., Tsatalas, C., Marcus, R. E., Goolden, A. W., Gordon-Smith, E. C., 1986. Bone marrow transplantation for patients with chronic myeloid leukaemia: T-cell depletion with Campath-1 reduces the incidence of graft-versus-host disease but may increase the risk of leukaemic relapse. Bone Marrow Transplant. 1, 53-66.
Bae, E. A., Seo, H., Kim, I. K., Jeon, I., Kang, C. Y., 2019. Roles of NKT cells in cancer immunotherapy. Arch. Pharm. Res. 42, 543-548. https://doi.org/10.1007/s12272-019-01139-8
Ball, L. M., Egeler, R. M., 2008. Acute GvHD: Pathogenesis and classification. Bone Marrow Transplant. 41, 58-64. https://doi.org/10.1038/bmt.2008.56
Bendelac, A., Savage, P. B., Teyton, L., 2007. The Biology ofNKT Cells. Annu. Rev. Immunol. 25, 297-336. https://doi.org/10.1146/annurev.immunol.25.022106.141711
Brennan, P. J., Brigl, M., Brenner, M. B., 2013. Invariant natural killer T cells: an innate activation scheme linked to diverse effector functions. Nat. Rev. Immunol. 13, 101-117. https://doi.org/10.1038/nri3369
Brigl, M., Brenner, M. B., 2004. CD1: antigen presentation and T cell function. Annu. Rev. Immunol. 22, 817-890. https://doi.org/10.1146/annurev.immunol.22.012703.104608
Chaidos, A., Patterson, S., Szydlo, R., Chaudhry, M. S., Dazzi, F., Kanfer, E., McDonald, D., Marin, D., Milojkovic, D., Pavlu, J., Davis, J., Rahemtulla, A., Rezvani, K., Goldman, J., Roberts, I., Apperley, J., Karadimitris, A., 2012. Graft invariant natural killer T-cell dose predicts risk of acute graft-versus-host disease in allogeneic hematopoietic stem cell transplantation. Blood 119, 5030-5036. https://doi.org/10.1182/blood-2011-11-389304
Chakraverty, R., Sykes, M., 2007. The role of antigen-presenting cells in triggering graft-versus-host disease and graft-versus-leukemia. Blood 110, 9-17. https://doi.org/10.1182/blood-2006-12-022038
Cohen, N. R., Garg, S., Brenner, M. B., 2009. Antigen Presentation by CD1 Lipids, T Cells, and NKT Cells in Microbial Immunity. Adv. Immunol. 102. 1-94. https://doi.org/10.1016/S0065-2776(09)01201-2
Coman, T., Rossignol, J., D'Aveni, M., Fabiani, B., Dussiot, M., Rignault, R., Babdor, J., Bouillé, M., Herbelin, A., Cote, F., Moura, I. C., Hermine, O., Rubio, M. T., 2018. Human CD4− invariant NKT lymphocytes regulate graft versus host disease. Oncoimmunology 7, 1-10. https://doi.org/10.1080/2162402X.2018.1470735
Cortesi, F., Delfanti, G., Grilli, A., Calcinotto, A., Gorini, F., Pucci, F., Luciano, R., Grioni, M., Recchia, A., Benigni, F., Briganti, A., Salonia, A., De Palma, M., Bicciato, S., Doglioni, C., Bellone, M., Casorati, G., Dellabona, P., 2018. Bimodal CD40/Fas-Dependent Crosstalk between iNKT Cells and Tumor-Associated Macrophages Impairs Prostate Cancer Progression. Cell Rep. 22, 3006-3020. https://doi.org/10.1016/j.celrep.2018.02.058
Ferrara, J. L. M., Levine, J. E., Reddy, P., Holler, E., 2009. Graft-versus-host disease. Lancet (London, England) 373, 1550-1561. https://doi.org/10.1016/S0140-6736(09)60237-3
Fujii, S.-I., Shimizu, K., Okamoto, Y., Kunii, N., Nakayama, T., Motohashi, S., Taniguchi, M., 2013. NKT cells as an ideal anti-tumor immunotherapeutic. Front. Immunol. 4, 409. https://doi.org/10.3389/fimmu.2013.00409
Godfrey, D. I., Berzins, S. P., 2007. Control points in NKT-cell development. Nat. Rev. Immunol. 7, 505-518. https://doi.org/10.1038/nri2116
Gooptu, M., Antin, J. H., 2021. GVHD Prophylaxis 2020. Front. Immunol. 12, 1-13. https://doi.org/10.3389/fimmu.2021.605726
Gorini, F., Azzimonti, L., Delfanti, G., Scarf6, L., Scielzo, C., Bertilaccio, M. T., Ranghetti, P., Gulino, A., Doglioni, C., Di Napoli, A., Capri, M., Franceschi, C., Caligaris-Cappio, F., Ghia, P., Bellone, M., Dellabona, P., Casorati, G., de Lalla, C., 2017. Invariant NKT cells contribute to chronic lymphocytic leukemia surveillance and prognosis. Blood 129, 3440-3451. https://doi.org/10.1182/blood-2016-11-751065
Gribben, J. G., O'Brien, S., 2011. Update on therapy of chronic lymphocytic leukemia. J. Clin. Oncol. Off. J. Am. Soc. Clin. Oncol. 29, 544-550. https://doi.org/10.1200/JCO.2010.32.3865
Haraguchi, K., Takahashi, T., Hiruma, K., Kanda, Y., Tanaka, Y., Ogawa, S., Chiba, S., Miura, O., Sakamaki, H., Hirai, H., 2004. Recovery of Va.24+ NKT cells after hematopoietic stem cell transplantation. Bone Marrow Transplant. 34, 595-602. https://doi.org/10.1038/sj.bmt.1704582
Hill, G. R., Betts, B. C., Tkachev, V., Kean, L. S., Blazar, B R., 2021. Current Concepts and Advances in Graft-Versus-Host Disease Immunology. Annu. Rev. Immunol. 39, 19-49. https://doi.org/10.1146/annurev-immunol-102119-073227
Horowitz, M. M., Gale, R. P., Sondel, P. M., Goldman, J. M., Kersey, J., Kolb, H. J., Rimm, A. A., Ringden, O., Rozman, C., Speck, B., Truitt, R. L., Zwaan, F. E., Bortin, M. M., 1990. Graft-versus-leukemia reactions after bone marrow transplantation. Blood 75, 555-562. https://doi.org/10.1182/blood.v75.3.555.555
Huang, F., Cao, F. L., Zheng, S. G., 2018. Update of humanized animal disease models in studying Graft-versus-host disease. Hum. Vaccin. Immunother. 14, 2618-2623. https://doi.org/10.1080/21645515.2018.1512454
Huijskens, M.J.A.J., Walczak, M., Koller, N., Briede, J. J., Senden-Gijsbers, B.L.M.G., Schnijderberg, M. C., Bos, G. M. J., Germeraad, W. T. V, 2014. Technical advance: ascorbic acid induces development of double-positive T cells from human hematopoietic stem cells in the absence of stromal cells. J. Leukoc. Biol. 96, 1165-1175. https://doi.org/10.1189/jlb.1TA0214-121RR
Iriguchi, S., Yasui, Y., Kawai, Y., Arⁱma, S., Kunitomo, M., Sato, T., Ueda, T., Minagawa, A., Mishima, Y., Yanagawa, N., Baba, Y., Miyake, Y., Nakayama, K., Takiguchi, M., Shinohara, T., Nakatsura, T., Yasukawa, M., Kassai, Y., Hayashi, A., Kaneko, S., 2021. A clinically applicable and scalable method to regenerate T-cells from iPSCs for off-the-shelf T-cell immunotherapy. Nat. Commun. 12, 430. https://doi.org/10.1038/s41467-020-20658-3
Janakiram, N. B., Mohammed, A., Bryant, T., Ritchie, R., Stratton, N., Jackson, L., Lightfoot, S., Benbrook, D. M., Asch, A. S., Lang, M. L., Rao, C. V, 2017. Loss of natural killer T cells promotes pancreatic cancer in LSL-Kras(G12D/+) mice. Immunology 152, 36-51. https://doi.org/10.1111/imm.12746
Jardine, L., Cytlak, U., Gunawan, M., Reynolds, G., Green, K., Wang, X.-N., Pagan, S., Paramitha, M., Lamb, C. A., Long, A. K., Hurst, E., Nair, S., Jackson, G. H., Publicover, A., Bigley, V., Haniffa, M., Simpson, A. J., Collin, M., 2020. Donor monocyte-derived macrophages promote human acute graft-versus-host disease. J. Clin. Invest. 130, 4574-4586. https://doi.org/10.1172/JCI133909
King, L. A., Lameris, R., de Gruijl, T. D., van der Viet, H. J., 2018. CD1d-Invariant Natural Killer T Cell-Based Cancer Immunotherapy: a-Galactosylceramide and Beyond. Front. Immunol. 9. https://doi.org/10.3389/finnu.2018.01519
King, M. A., Covassin, L., Brehm, M. A., Racki, W., Pearson, T., Leif, J., Laning, J., Fodor, W., Foreman, O., Burzenski, L., Chase, T. H., Gott, B., Rossini, A. A., Bortell, R., Shultz, L. D., Greiner, D. L., 2009. Human peripheral blood leucocyte non-obese diabetic-severe combined immunodeficiency interleukin-2 receptor gamma chain gene mouse model of xenogeneic graft-versus-host-like disease and the role of host major histocompatibility complex. Clin. Exp. Immunol. 157, 104-118. https://doi.org/10.1111/j.1365-2249.2009.03933.x
Kohrt, H. E., Turnbull, B. B., Heydari, K., Shizuru, J. A., Laport, G. G., Miklos, D. B., Johnston, L. J., Arai, S., Weng, W.-K., Hoppe, R. T., Lavori, P. W., Blume, K. G., Negrin, R. S., Strober, S., Lowsky, R., 2009. TLI and ATG conditioning with low risk of graft-versus-host disease retains antitumor reactions after allogeneic hematopoietic cell transplantation from related and unrelated donors. Blood 114, 1099-1109. https://doi.org/10.1182/blood-2009-03-211441
Krijgsman, D., Hokland, M., Kuppen, P. J. K., 2018. The role of natural killer T cells in cancer-A phenotypical and functional approach. Front. Immunol. 9. https://doi.org/10.3389/fimmu.2018.00367
Kronenberg, M., 2005. Toward an understanding ofNKT cell biology: progress and paradoxes. Annu. Rev. Immunol. 23, 877-900. https://doi.org/10.1146/annurev.immunol.23.021704.115742
Kumar, A., Suryadevara, N., Hill, T. M., Bezbradica, J. S., Van Kaer, L.. Joyce, S., 2017. Natural Killer T Cells: An Ecological Evolutionary Developmental Biology Perspective. Front. Immunol. 8, 1858. https://doi.org/10.3389/fimmu.2017.01858
Lan, F., Zeng, D., Higuchi, M., Higgins, J. P., Strober, S., 2003. Host conditioning with total lymphoid irradiation and antithymocyte globulin prevents graft-versus-host disease: the role of CD1-reactive natural killer T cells. Biol. blood marrow Transplant. J. Am. Soc. Blood Marrow Transplant. 9, 355-363. https://doi.org/10.1016/s1083-8791(03)00108-3
Lan, F., Zeng, D., Higuchi, M., Huie, P., Higgins, J. P., Strober, S., 2001. Predominance of NK1.1+TCRαβ+or DX5+TCRαβ+T Cells in Mice Conditioned with Fractionated Lymphoid Irradiation Protects Against Graft-Versus-Host Disease: “Natural Suppressor” Cells. J. Immunol. 167, 2087-2096. https://doi.org/10.4049/jimmunol.167.4.2087
Lantz, O., Bendelac, A., 1994. An invariant T cell receptor alpha chain is used by a unique subset of major histocompatibility complex class 1-specific CD4+ and CD4-8- T cells in mice and humans. J. Exp. Med. 180, 1097-1106. https://doi.org/10.1084/jem.180.3.1097
Li, Y.-R., Brown, J., Yu, Y., Lee, D., Zhou, K., Dunn, Z. S., Hon, R., Wilson, M., Kramer, A., Zhu, Y., Fang, Y., Yang, L., 2022. Targeting Immunosuppressive Tumor-Associated Macrophages Using Innate T Cells for Enhanced Antitumor Reactivity. Cancers. https://doi.org/10.3390/cancers14112749
Li, Y.-R., Dunn, Z. S., Zhou, Y., Lee, D., Yang, L., 2021a. Development of Stem Cell-Derived Immune Cells for Off-the-Shelf Cancer Immunotherapies. Cells 10. https://doi.org/10.3390/cells10123497
Li, Y.-R., Zhou, Y., Kim, Y. J., Zhu, Y., Ma, F., Yu, J., Wang, Y.-C., Chen, X., Li, Z., Zeng, S., Wang, Xi, Lee, D., Ku, J., Tsao, T., Hardoy, C., Huang, J., Cheng, D., Montel-Hagen, A., Seet, C. S., Crooks, G. M., Larson, S. M., Sasine, J. P., Wang, Xiaoyan, Pellegrini, M., Ribas, A., Kohn, D. B., Witte, O., Wang, P., Yang, L., 2021b. Development of allogeneic HSC-engineered iNKT cells for off-the-shelf cancer immunotherapy. Cell reports. Med. 2, 100449. https://doi.org/10.1016/j.xcrm.2021.100449
Li, Y. R., Dunn, Z. S., Jr, G. G., Carmona, C., Zhou, Y., Lee, D., Yu, J., Huang, J., Kim, J. T., Arumugaswami, V., Wang, P., Yang, L., 2022. Development of off—the—shelf hematopoietic stem cell—engineered invariant natural killer T cells for COVID—19 therapeutic intervention. Stem Cell Res. Ther. 1-15. https://doi.org/10.1186/s13287-022-02787-2
Lowsky, R., Takahashi, T., Liu, Y. P., Dejbakhsh-Jones, S., Grumet, F. C., Shizuru, J. A., Laport, G. G., Stockerl-Goldstein, K. E., Johnston, L. J., Hoppe, R. T., Bloch, D. A., Blume, K. G., Negrin, R. S., Strober, S., 2005. Protective conditioning for acute graft-versus-host disease. N. Engl. J. Med. 353, 1321-1331. https://doi.org/0.1056/NEJMoa050642
Malard, F., Huang, X. J., Sim, J. P. Y., 2020. Treatment and unmet needs in steroid-refractory acute graft-versus-host disease. Leukemia 34, 1229-1240. https://doi.org/10.1038/s41375-020-0804-2
Mavers, M., Maas-Bauer, K., Negrin, R. S., 2017. Invariant Natural Killer T Cells As Suppressors of Graft-versus-Host Disease in Allogeneic Hematopoietic Stem Cell Transplantation. Front. Immunol. 8, 900. https://doi.org/10.3389/fimmu.2017.00900
Montel-Hagen, A., Seet, C. S., Li, S., Chick, B., Zhu, Y., Chang, P., Tsai, S., Sun, V., Lopez, S., Chen, H. C., He, C., Chin, C. J., Casero, D., Crooks, G. M., 2019. Organoid-Induced Differentiation of Conventional T Cells from Human Pluripotent Stem Cells. Cell Stem Cell 24, 376-389.e8. https://doi.org/10.1016/j.stem.2018.12.O11
Pabst, C., Schirutschke, H., Ehninger, G., Bornhauser, M., Platzbecker, U., 2007. The graft content of donor T cells expressing gamma delta TCR+ and CD4+foxp3+predicts the risk of acute graft versus host disease after transplantation of allogeneic peripheral blood stem cells from unrelated donors. Clin. cancer Res. an Off. J. Am. Assoc. Cancer Res. 13, 2916-2922. https://doi.org/10.1158/1078-0432.CCR-06-2602
Passweg, J. R., Baldomero, H., Chabannon, C., Basak, G. W., Corbacioglu, S., Duarte, R., Dolstra, H., Lankester, A. C., Mohty, M., Montoto, S., Peffault de Latour, R., Snowden, J. A., Styczynski, J., Yakoub-Agha, I., Kröger, N., 2020. The EBMT activity survey on hematopoietic-cell transplantation and cellular therapy 2018: CAR-T's come into focus. Bone Marrow Transplant. 55, 1604-1613. https://doi.org/10.1038/s41409-020-0826-4
Pillai, A. B., George, T. I., Dutt, S., Teo, P., Strober, S., 2007. Host NKT Cells Can Prevent Graft-versus-Host Disease and Permit Graft Antitumor Activity after Bone Marrow Transplantation. J. Immunol. 178, 6242-6251. https://doi.org/10.4049/jimmunol.178.10.6242
Piper, C., Zhou, V., Komorowski, R., Szabo, A., Vincent, B., Serody, J., Alegre, M.-L., Edelson, B. T., Taneja, R., Drobyski, W. R., 2020. Pathogenic Bhlhe40+ GM-CSF+ CD4+ T cells promote indirect alloantigen presentation in the GI tract during GVHD. Blood 135, 568-581. https://doi.org/10.1182/blood.2019001696
Ramachandran, V., Kolli, S. S., Strowd, L. C., 2019. Review of Graft-Versus-Host Disease. Dermatol. Clin. 37, 569-582. https://doi.org/10.1016/j.det.2019.05.014
Rubio, M.-T., Moreira-Teixeira, L., Bachy, E., Bouillie, M., Milpied, P., Coman, T., Suarez, F., Marcais, A., Sibon, D., Buzyn, A., Caillat-Zucman, S., Cavazzana-Calvo, M., Varet, B., Dy, M., Hermine, O., Leite-de-Moraes, M., 2012. Early posttransplantation donor-derived invariant natural killer T-cell recovery predicts the occurrence of acute graft-versus-host disease and overall survival. Blood 120, 2144-2154. https://doi.org/i0.1182/blood-2012-01-404673
Rubio, M. T., Bouillie, M., Bouazza, N., Coman, T., Trebeden-Negre, H., Gomez, A., Suarez, F., Sibon, D., Brignier, A., Paubelle, E., Nguyen-Khoc, S., Cavazzana, M., Lantz, O., Mohty, M., Urien, S., Hermine, O., 2017. Pre-transplant donor CD4 —Invariant NKT cell expansion capacity predicts the occurrence of acute graft-versus-host disease. Leukemia 31, 903-912. https://doi.org/10.1038/leu.2016.281
Schneidawind, D., Baker, J., Pierini, A., Buechele, C., Luong, R. H., Meyer, E. H., Negrin, R. S., 2015. Third-party CD4⁺ invariant natural killer T cells protect from murine GVHD lethality. Blood 125, 3491-3500. https://doi.org/10.1182/blood-2014-11-612762
Schneidawind, D., Pierini, A., Alvarez, M., Pan, Y., Baker, J., Buechele, C., Luong, R. H., Meyer, E. H., Negrin, R. S., 2014. CD4+ invariant natural killer T cells protect from murine GVHD lethality through expansion of donor CD4+CD25+FoxP3+regulatory T cells. Blood 124, 3320-3328. https://doi.org/10.1182/blood-2014-05-576017
Schroeder, M. A., DiPersio, J. F., 2011. Mouse models of graft-versus-host disease: Advances and limitations. DMM Dis. Model. Mech. 4, 318-333. https://doi.org/i0.1242/dmm.006668
Seet, C. S., He, C., Bethune, M. T., Li, S., Chick, B., Gschweng, E. H., Zhu, Y., Kim, K., Kohn, D. B., Baltimore, D., Crooks, G. M., Montel-Hagen, A., 2017. Generation of mature T cells from human hematopoietic stem and progenitor cells in artificial thymic organoids. Nat. Methods 14, 521-530. https://doi.org/10.1038/nmeth.4237
Shimabukuro-Vornhagen, A., Hallek, M. J., Storb, R. F., von Bergwelt-Baildon, M. S.. 2009. The role of B cells in the pathogenesis of graft-versus-host disease. Blood 114, 4919-4927. https://doi.org/10.1182/blood-2008-10-161638
Shlomchik, W. D., 2007. Graft-versus-host disease. Nat. Rev. Immunol. 7, 340-352. https://doi.org/10.1038/nri2000
Shukla, S., Langley, M. A., Singh, J., Edgar, J. M., Mohtashami, M., Znniga-Pflucker, J. C., Zandstra, P. W., 2017. Progenitor T-cell differentiation from hematopoietic stem cells using Delta-like-4 and VCAM-1. Nat. Methods 14, 531-538. https://doi.org/10.1038/nmeth.4258
Shultz, L. D., Ishikawa, F., Greiner, D. L., 2007. Humanized mice in translational biomedical research. Nat. Rev. Immunol. 7, 118-130. https://doi. org/10.1038/nri2017
Smith, D. J., Liu, S., Ji, S., Li, B., McLaughlin, J., Cheng, D., Witte, O. N., Yang, L., 2015. Genetic engineering of hematopoietic stem cells to generate invariant natural killer T cells. Proc. Natl. Acad. Sci. U.S.A 112, 1523-1528. https://doi.org/10.1073/pnas.1424877112
Song, L., Asgharzadeh, S., Salo, J., Engell, K., Wu, H., Sposto, R., Ara, T., Silverman, A. M., DeClerck, Y. A., Seeger, R. C., Metelitsa, L. S., 2009. Valpha24-invariant NKT cells mediate antitumor activity via killing of tumor-associated macrophages. J. Clin. Invest. 119, 1524-1536. https://doi.org/10.1172/JCI37869
Taniguchi, M., Harada, M., Kojo, S., Nakayama, T., Wakao, H., 2003. The regulatory role of Valpha14 NKT cells in innate and acquired immune response. Annu. Rev. Immunol. 21, 483-513. https://doi.org/10.1146/annurev.immunol.21.120601.141057
Themeli, M., Kloss, C. C., Ciriello, G., Fedorov, V. D., Pema, F., Gonen, M., Sadelain, M., 2013. Generation of tumor-targeted human T lymphocytes from induced pluripotent stem cells for cancer therapy. Nat. Biotechnol. 31, 928-933. https://doi.org/10.1038/nbt.2678
Tugues, S., Amorim, A., Spath, S., Martin-Blondel, G., Schreiner, B., De Feo, D., Lutz, M., Guscetti, F., Apostolova, P., Haftmann, C., Hasselblatt, P., Nn5ez, N. G., Hottiger, M. O., van den Broek, M., Manz, M. G., Zeiser, R., Becher, B., 2018. Graft-versus-host disease, but not graft-versus-leukemia immunity, is mediated by GM-CSF-licensed myeloid cells. Sci. Transl. Med. 10. https://doi.org/10.1126/scitranslmed.aat8410
Van Kaer, L., Parekh, V. V., Wu, L., 2011. Invariant natural killer T cells: Bridging innate and adaptive immunity. Cell Tissue Res. 343, 43-55. https://doi.org/10.1007/s00441-010-1023-3
Wolf, D., Wolf, A.M., Fong, D., Rumpold, H., Strasak, A., Clausen, J., Nachbaur, D., 2007. Regulatory T-cells in the graft and the risk of acute graft-versus-host disease after allogeneic stem cell transplantation. Transplantation 83, 1107-1113. https://doi.org/10.1097/01.tp.0000260140.04815.77
Wu, T., Young, J.S., Johnston, H., Ni, X., Deng, R., Racine, J., Wang, M., Wang, A., Todorov, I., Wang, J., Zeng, D., 2013. Thymic damage, impaired negative selection, and development of chronic graft-versus-host disease caused by donor CD4+ and CD8+ T cells. J. Immunol. 191, 488-499. https://doi.org/10.4049/jimmunol.1300657
Yamasaki, S., Henzan, H., Ohno, Y., Yamanaka, T., lino, T., Itou, Y., Kuroiwa, M., Maeda, M., Kawano, N., Kinukawa, N., Miyamoto, T., Nagafuji, K., Shimoda, K., Inaba, S., Hayashi, S., Taniguchi, S., Shibuya, T., Gondo, H., Otsuka, T., Harada, M., 2003. Influence of transplanted dose of CD56+ cells on development of graft-versus-host disease in patients receiving G-CSF-mobilized peripheral blood progenitor cells from HLA-identical sibling donors. Bone Marrow Transplant. 32, 505-510. https://doi.org/10.1038/sj.bmt.1704165
Zeng, D., Lewis, D., Dejbakhsh-Jones, S., Lan, F., Garcia-Ojeda, M., Sibley, R., Strober, S., 1999. Bone marrow NK1.1(−) and NK1.1(+) T cells reciprocally regulate acute graft versus host disease. J. Exp. Med. 189, 1073-1081. https://doi.org/10.1084/jem.189.7.1073
Zhou, Y., Li, Y.-R., Zeng, S., Yang, L., 2021. Methods for Studying Mouse and Human Invariant Natural Killer T Cells. Methods Mol. Biol. 2388, 35-57. https://doi.org/10.1007/978-1-0716-1775-5_4
Zhu, Y., Smith, D.J., Zhou, Y., Li, Y. R., Yu, J., Lee, D., Wang, Y. C., Di Biase, S., Wang, Xi, Hardoy, C., Ku, J., Tsao, T., Lin, L. J., Pham, A. T., Moon, H., McLaughlin, J., Cheng, D., Hollis, R. P., Campo-Fernandez, B., Urbinati, F., Wei, L., Pang, L., Rezek, V., Berent-Maoz, B., Macabali, M. H., Gjertson, D., Wang, Xiaoyan, Galic, Z., Kitchen, S. G., An, D. S., Hu-Lieskovan, S., Kaplan-Lefko, P. J., De Oliveira, S. N., Seet, C. S., Larson, S. M., Forman, S. J., Heath, J. R., Zack, J. A., Crooks, G. M., Radu, C. G., Ribas, A., Kohn, D. B., Witte, O. N., Yang, L., 2019. Development of Hematopoietic Stem Cell-Engineered Invariant Natural Killer T Cell Therapy for Cancer. Cell Stem Cell 25, 542-557.e9. https://doi.org/10.1016/j.stem.2019.08.004

All publications mentioned herein (e.g. the references numerically listed above and U.S. Patent Applications having serial numbers U.S. Pat. No. 10,927,160, U.S. Ser. No. 17/251,118 and U.S. Ser. No. 17/618,240) are incorporated herein by reference to disclose and describe aspects, methods and/or materials in connection with the cited publications. Certain aspects and embodiments of the invention are found in the disclosure of the Appendix submitted herewith, which is herein incorporated by reference.

1. A method of inhibiting or treating a graft versus host disease in a subject in need thereof, comprising administering to said subject a therapeutically effective amount of allogeneic HSC-engineered human iNKT cells such that graft versus host disease is inhibited or treated in the subject.

2. The method of claim 1, wherein the graft versus host disease is selected from the group consisting of acute graft-versus-host-disease (aGVHD) and chronic graft-versus-host-disease (cGVHD).

3. The method of claim 1, wherein the subject has been diagnosed with hematologic malignancy.

4. The method of claim 1, wherein the subject has undergone or will undergo an allogeneic hematopoietic stem cell transplantation procedure.

5. The method of claim 4, wherein the subject is administered the allogeneic HSC-engineered human iNKT cells at the time the subject undergoes the allogeneic hematopoietic stem cell transplantation procedure.

6. The method of claim 5, wherein the subject is administered allogeneic HSC-engineered human iNKT cells mixed with allogeneic hematopoietic stem cells.

7. The method of claim 1, wherein the subject is administered at least 1×10⁶ allogeneic HSC-engineered human iNKT cells.

8. The method of claim 1, wherein the subject is administered at least 0.031×10⁶ cells/kg of body weight of the allogeneic HSC-engineered human iNKT cells.

9. The method of claim 1, wherein the engineered iNKT cells comprise one or more exogenous nucleic acids transduced therein, wherein:
the one or more exogenous nucleic acids comprise a Vα24-Jα18 iNKT cell receptor gene; and/or
the one or more exogenous nucleic acids comprise a classical α or β T cell receptor gene.

10. A method of depleting allogeneic CD14⁺ myeloid cells from a subject transfused with allogenic leukocytes including the allogeneic CD14⁺ myeloid cells, the method comprising administering to said subject amounts of allogeneic HSC-engineered human iNKT cells sufficient to target the allogeneic CD14⁺ myeloid cells in the subject, thereby depleting the HSC-engineered human iNKT cells in the subject.

11. The method of claim 10, wherein the allogeneic HSC-engineered human iNKT cells comprise one or more exogenous nucleic acids, wherein:
the one or more exogenous nucleic acids comprise a Vα24-Jα18 iNKT cell receptor gene; and/or
the one or more exogenous nucleic acids comprise a classical α or β T cell receptor gene.

12. The method of claim 10, wherein the subject is administered at least 1 λ10⁶ allogeneic HSC-engineered human iNKT cells.

13. The method of claim 10, wherein the subject is administered allogeneic HSC-engineered human iNKT cells at the time that the subject is transfused with the allogenic leukocytes.

14. The method of claim 10, wherein the subject has been diagnosed with hematologic malignancy.

15. A method of inhibiting or suppressing expansion of Th1-type pathogenic donor T cells in a subject treated with allogenic T cells in a therapeutic regimen, the method comprising administering to said subject amounts of allogeneic HSC-engineered human iNKT cells sufficient to inhibit or suppress the expansion of Th1-type pathogenic donor T cells in the subject.

16. The method of claim 15, wherein the subject is administered at least 1×10⁶ allogeneic HSC-engineered human iNKT cells.

17. The method of claim 15, wherein the subject is administered allogeneic HSC-engineered human iNKT cells at the time that the subject is treated with the allogenic T cells in the therapeutic regimen.

18. The method of claim 15, wherein the allogeneic HSC-engineered human iNKT cells:
are derived from hematopoetic stem cells transduced with one or more exogenous nucleic acids comprising a Vα24-Jα18 T cell receptor gene receptor gene.

19. The method of claim 18, wherein the subject is selected to be a patient diagnosed with hematologic malignancy.

20. The method of claim 19, wherein the subject is administered at least 0.031×10⁶ cells/kg of body weight of the allogeneic HSC-engineered human iNKT cells.
